(19) United States
(12) Patent Application Publication
(10) Pub. No.: US 2013/0202672 A1
(43) Pub. Date: Aug. 8, 2013

(54) FIBER-ASSEMBLED TISSUE CONSTRUCTS

(71) Applicants: Andrew Chwee Aun Wan, Singapore (SG); Meng Fatt Leong, Singapore (SG); Jackie Y. Ying, Singapore (SG); Jerry Kah Chin Toh, Singapore (SG)

(72) Inventors: Andrew Chwee Aun Wan, Singapore (SG); Meng Fatt Leong, Singapore (SG); Jackie Y. Ying, Singapore (SG); Jerry Kah Chin Toh, Singapore (SG)

(73) Assignee: Singapore Agency for Science, Technology and Research Act

(21) Appl. No.: 13/579,920

(22) PCT Filed: Feb. 19, 2010
(86) PCT No.: PCT/SG10/00062

§ 371 Date: Dec. 28, 2012

(51) Int. Cl. A61L 27/54; A61L 27/20; A61L 27/24

(52) U.S. Cl. CPC A61L 27/54; A61L 27/24; A61L 27/20
USPC 424/422; 424/93.1; 424/93.7; 156/180

(57) ABSTRACT

The present invention relates to a fiber-assembled tissue construct comprising at least one sinusoid unit, the unit comprising at least two polymeric fibers arranged in a sinusoid structure and fused together, each of said fibers comprising a porous matrix supporting biological components encapsulated in the fiber, wherein the biological components are patterned in three-dimensions within the construct.

### TECHNICAL FIELD

The invention relates to fibers and the assembly of constructs from the same. More specifically, the invention relates to the assembly of fibers into three dimensional constructs suitable for biological applications including tissue engineering, drug testing, and the analysis of cells.

### BACKGROUND

Engineering complex tissues involves organizing multiple cell types in a precise three-dimensional (3D) ultrastructure. The size and viability of the engineered tissue are constrained by the availability and accessibility of vasculatures that provide efficient transportation networks for nutrients and waste. In order to build such intricate architectural structures, one has to design hierarchical constructs ranging from microscale single cell units to macroscale 3D orchestrated tissues. Thus, the engineering of complex organs necessitates the development of tools that can precisely pattern the various cell types in 3D.

Several ways of patterning cells in three dimensions have been expounded and investigated. Organ plotting, which involves the robot-assisted dispensing of cells in defined patterns via a nozzle and which calls upon the inherent nature of cells to self-assemble, is based on the premise that an acellular scaffold component is unnecessary. Cell sheet technology is another scaffold-free cell patterning method which also relies on cellular self-assembly to form a construct that may be vascularised. On the other hand, the use of dielectrophoretic patterning and laser-guided direct writing leverages on the precise placement of cells or ‘microphases’ within a 3D hydrogel matrix.

A number of current technologies (e.g. organ printing and 3D cell plotting) lack the capability to meet all of three main requirements of cell patterning: namely, high resolution, high cell density and three-dimensionality. Moreover, cell patterning is done in series and is thus time consuming. Although technologies are available that provide increased resolution (e.g. laser-guided direct writing) they have not been extended to robust 3D structures which are important for clinically relevant constructs. In addition, vascularisation afforded by current technologies is in many cases suboptimal.

A need exists for improved constructs capable of micropatterning cells and other biological materials at high resolution in a three-dimensional environment.

### SUMMARY OF THE INVENTION

The present invention provides methods for the assembly of fibers containing encapsulated cells and/or other biological materials into a three-dimensional hierarchical construct. The construct provides a means to create a customisable three-dimensional micropatterned environment at high resolution.

In a first aspect, the invention provides a fiber-assembled tissue construct comprising at least one sinusoid unit, the unit comprising at least two polymeric fibers arranged in a sinusoid structure and fused together, each fiber comprising a porous matrix supporting biological components encapsulated in the fiber, wherein the biological components are patterned in three-dimensions within the construct.

In one embodiment of the first aspect, at least one of the biological components is an encapsulated cell.

In one embodiment of the first aspect, at least one of the polymeric fibers is a multi-component fiber, the multi-component fiber comprising at least two spatially defined internal domains.

In one embodiment of the first aspect, the multi-component fiber comprises a first internal domain and a second internal domain, the first internal domain comprising at least one component that is absent in the second internal domain.

In one embodiment of the first aspect, the sinusoid structure comprises a first polymeric fiber and a second polymeric fiber, the first polymeric fiber comprising at least one component that is absent in the second polymeric fiber.

In one embodiment of the first aspect, the at least one component is a specific type of cell, biologic or chemical component.

In one embodiment of the first aspect, at least one of said polymeric fibers forms a central fiber, and at least one of the polymeric fibers forms an outer fiber wrapped around said central fiber.

In one embodiment of the first aspect, at least one of the polymeric fibers comprises a biological or chemical component selected from the group consisting of extracellular matrix proteins, cytoskeletal proteins, cell adhesion proteins, hormones, growth factors, angiogenic factors, amino acids, nucleic acids, galactose ligands, drugs, and mixtures thereof.

In one embodiment of the first aspect, the sinusoid structure comprises a central fiber, and the central fiber comprises one or more of encapsulated endothelial cells, encapsulated epithelial cells or encapsulated neurons.

In one embodiment of the first aspect, the sinusoid structure comprises a central fiber and an outer fiber wrapped around the central fiber, the central fiber comprising encapsulated endothelial cells and said outer fiber comprising encapsulated hepatocytes.

In one embodiment of the first aspect, the sinusoid structure comprises a central fiber and an outer fiber wrapped around the central fiber, the central fiber comprising encapsulated epithelial cells and the outer fiber comprising encapsulated fibroblasts.

In one embodiment of the first aspect, the sinusoid structure comprises a central fiber and an outer fiber wrapped around the central fiber, the central fiber comprising encapsulated neurons and the outer fiber comprising encapsulated Schwann cells and/or encapsulated oligodendrocytes.

In a second aspect, the invention provides a method for producing a three-dimensional fiber-assembled tissue construct comprising at least one sinusoid unit, the method comprising the steps of:

(a) dispensing at least two polyionic solutions in separate locations on a first template;

(b) drawing a separate nascent polymeric fiber from each polyionic solution, wherein a first end of each nascent fiber remains attached to the first template and a second end of each nascent fiber remains attached to an opposing second template;

(c) rotating either or both templates to contact each fiber at a common fusion point; and

(d) fusing contacting fibers together to provide a sinusoid unit, wherein the fusing comprises:
(i) applying a fusing reagent to the fusion point and upwardly drawing each fibre such that the reagent travels downwardly along contacting fibers; or
(ii) continuing rotation of either or both templates causing fusion by compressive force.

In one embodiment of the second aspect, the method comprises the additional step of fusing two or more sinusoid units together.

In one embodiment of the second aspect, the fusing of two or more sinusoid units is performed by spooling sinusoid units and fusing them together with a fusing reagent.

In one embodiment of the second aspect, the fusing reagent is selected from the group consisting of polyanionic polymers, polycationic polymers, multivalent cations, multivalent anions, or mixtures thereof.

In one embodiment of the second aspect, at least one of the polymeric fibers is a multi-component fiber, the multi-component fiber comprising at least two spatially defined internal domains.

In one embodiment of the second aspect, the sinusoid unit comprises at least one central fiber and at least one outer fiber wrapped around the central fiber.

In one embodiment of the second aspect, at least one of the polymeric fibers comprises a cell.

In one embodiment of the second aspect, the sinusoid unit comprises a central fiber, and the central fiber comprises one or more of encapsulated endothelial cells, encapsulated epithelial cells or encapsulated neurons.

In one embodiment of the second aspect, the sinusoid unit comprises at least one fiber comprising a biological or chemical component selected from the group consisting of extracellular matrix proteins, cytoskeletal proteins, cell adhesion proteins, hormones, growth factors, angiogenic factors, amino acids, nucleic acids, galactose ligands, drugs, and mixtures thereof.

In one embodiment of the second aspect, the method is performed in a humidified chamber.

In one embodiment of the second aspect, biological components are micropatterned in three-dimensions in the sinusoid unit at a resolution of less than 50 μm.

In a third aspect, the invention provides a three-dimensional fiber-assembled tissue construct obtained by the method of the second aspect.

In a fourth aspect, the invention provides an apparatus for producing a three-dimensional fiber-assembled tissue construct comprising at least one sinusoid unit, the apparatus comprising:

(a) a first template comprising an upper surface suitable for the deposit of polyionic solutions;

(b) a drawing template comprising a lower surface opposing said upper surface of the first template, said lower surface comprising at least two protruding pointed tips; and

(c) an elongate shaft attached to the drawing template capable of upward and downward movement along its vertical axis;

wherein during use of the apparatus, nascent fibers are drawn from the polyionic solutions by upward movement of a protruding tip in contact with each solution and the sinusoid unit is formed by rotating either or both templates and fusing contacting fibers together.

In one embodiment of the fourth aspect, the apparatus further comprises a humidifying chamber housing each template and at least a portion of the elongate shaft.

In a fifth aspect, the invention provides a method for producing a multi-component fiber comprising at least two domains, the method comprising the steps of:

(i) arranging a series of at least three polyelectrolyte solutions on a surface, wherein the series comprises at least one solution flanked by adjacent solutions of opposite charge;

(ii) forming a series of at least two separate interfaces between opposing surfaces of oppositely charged adjacent polyelectrolyte solutions;

(iii) drawing a nascent fiber from each interface in an upward motion at a suitable rate until the nascent fibers fuse forming a single multi-component fiber.

In one embodiment of the fifth aspect, the multi-component fiber comprises two domains and the series comprises three polyelectrolyte solutions.

In one embodiment of the fifth aspect, the multi-component fiber comprises three domains and the series comprises five polyelectrolyte solutions.

In one embodiment of the fifth aspect, drawing a nascent fiber from each interface in an upward motion is conducted at a rate of between about 0.05 mm and 0.5 mm per second.

In one embodiment of the fifth aspect, biological components are micropatterned in three-dimensions within the multi-component fiber at a resolution of less than 50 μm.

In a sixth aspect, the invention provides a multi-component fiber obtained by the method of the fifth aspect.

### BRIEF DESCRIPTION OF THE DRAWINGS

A preferred embodiment of the present invention will now be described, by way of an example only, with reference to the accompanying drawings wherein:

FIG. 1 shows engineering of hierarchical tissue structures by fiber assembly. FIG. 1a is a schematic demonstrating how a micropatterned niche environment can be created by assembling primary (1°) biostructural units containing Cell A and Cell B and a multi-component fiber containing Cell C and Cell D in separate domains. Each secondary sinusoid structure(2°) consists of one central fiber containing Cell A surrounded by six other fibers, five of which contain Cell B and one multi-component fiber containing Cell C and Cell D in separate domains. This micropattem is repeated in the eventual tertiary construct (3°). *=individual niches within fibres; 1=Cell A; 2=Cell B; 3=Cell C; 4=Cell D.

FIG. 1b is a schematic of the process to achieve the sinusoid structure. Polyionic solutions containing bioactive components (cells, ECM proteins and growth factors) are placed on a template. Nine IPC fibers were drawn in parallel and brought to fuse at a single point by rotation of the template. Continuous upward drawing coupled with fusing by the sliding sodium alginate droplet forms the secondary sinusoid structure. 1: polyionic solutions localised on a template; 2-3: polyelectrolyte fibers drawn; 4-5: rotation brings green fibers to surround the red fibers; 6: dilute alginate solution fuses the fibers in place; 7: simultaneous drawing and fusing results in secondary sinusoid structure; 7: secondary sinusoid structure assembled to tertiary structure by spooling. s.s; secondary sinusoid structure.

FIG. 1c provides confocal micrographs of the biostructural units consisting of cells encapsulated in chitin-alginate fibers. Endothelial cells (EC) and hepatocyte cell line (HepG2) were fluorescently labeled orange and green, respectively. One EC-containing fiber (i) was surrounded by eight HepG2-containing fibers to form the sinusoid structure (ii). The tertiary structure (iii) that contains micropatterned cells was formed by rolling up the sinusoid structures. Bar (i)=100 μm; bar (ii)=100 μm; bar (iii)=100 μm.

FIGS. 2a and 2b are graphs indicative of the function of primary rat hepatocytes encapsulated in tertiary constructs over 7 days. FIG. 2a shows the percentage of normalised albumin secretion (y-axis) over days 0-7 (x-axis). FIG. 2b shows the percentage of normalised urea synthesis function (y-axis) over days 0-7 (x-axis). Dark shaded bar represents co-culture of hepatocytes with HUVEC. Light shaded bar represents hepatocyte monoculture.

FIG. 3 shows fluorescence micrographs of Live/Dead cell viability assays of cells in fiber assembled tertiary structure. FIG. 3a: Coronary artery smooth muscle cells (CASMC) at 10×; FIG. 3b: CASMC at 20×; FIG. 3c: Hepatocyte cell line (HepG2) at 10×; FIG. 3d: HepG2 at 20×.

FIG. 4 illustrates linear head pipette tip configurations for drawing multi interfacial polyelectrolyte complexation (MIPC) fiber.

FIGS. 5a and 5b illustrate linear head pipette tip configurations for drawing a 2-component fiber. Light (1, 3) and dark (2) regions denote oppositely charged polyelectrolytes.

FIGS. 6a and 6b illustrate linear head pipette tip configurations for drawing a 2-component fiber. Light (2, 4) and dark (1, 3, 5) regions denote oppositely charged polyelectrolytes.

FIG. 7a is a schematic showing a procedure for drawing a 2-component fiber from two interfaces.

FIG. 7b is a schematic showing a procedure for drawing a 3-component fiber from four interfaces.

FIG. 8 is a fluorescence micrograph showing the bead region of a 2-component (binary) MIPC fiber.

FIG. 9 is a fluorescence micrograph showing the bead region of a 3-component (ternary) MIPC fiber.

FIG. 10 is a fluorescence micrograph showing the fiber region of a 3-component (ternary) MIPC fiber.

FIG. 11 provides microscopy images of MC3T3 cells in 3-component (ternary) MIPC fiber after 0 days (FIG. 11a); 3 days (FIG. 11b), and 7 days (FIG. 11c). In FIGS. 11a and 11b, the original cell-free centre domain is defined by the black dashed lines. By Day 3 (FIG. 11b), cells have begun to form aggregates and the cell distribution is generally less well defined. By Day 7 (FIG. 11c), most of the cells have formed clusters and the three domains within the fiber are no longer defined. The approximate fiber perimeter in FIG. 11c is defined by the white dashes.

FIG. 12 provides a microscopy image showing a 3-component (ternary) MIPC comprising two primary hepatocyte domains flanking a central domain containing human umbilical vein endothelial cells (HUVEC).

FIG. 13 provides a microscopy image showing interactions between hepatocytes (HEP) and tube-forming endothelial cells (HUVEC) at the interface of two domains in a 3-component (ternary) fiber. *=HUVEC; **=HEP.

FIG. 14a provides a confocal micrograph showing rope-like structure formed by twisting four individually fluorescent labelled fibers without the center fiber. FIG. 14b provides a confocal micrograph showing a central fiber consisting of HUVEC (labelled fluorescence red) wrapped by twisting four outer fibers consisting of HepG2 (labelled fluorescence green) in a rope-like structure.

FIG. 15 shows an apparatus for the production of fibers in accordance with the invention.

FIGS. 16a-16c are microscopy images showing tubule morphogenesis of canine kidney epithelial cells (MDCK) cells cultured for 5 days. FIG. 16a shows a control construct with MDCK in single culture, FIG. 16b and FIG. 16c show MDCK cells form tubules when co-cultured with fibroblasts in a same construct, or with the fibroblasts attached at the bottom of the culture dish, respectively.

FIG. 17 is a schematic showing the fusion of the two nascent fibers drawn from a three droplet configuration to form a two component fiber.

FIG. 18 is a schematic showing a procedure for drawing a multi-component fiber from four interfaces. FIG. 18a shows a plan view of an alternative configuration for drawing multi-component fibers. Different shaded portions denote oppositely charged polyelectrolyte solutions; FIG. 18b shows the same configuration, with channels/grooves to spatially define the solutions; FIG. 18c shows a corresponding instrument to draw the multi-component fibers.

### DEFINITIONS

As used in this application, the singular faun “a”, “an” and “the” include plural references unless the context clearly dictates otherwise. For example, the term “a plant cell” also includes a plurality of plant cells.

As used herein, the term “comprising” means “including.” Variations of the word “comprising”, such as “comprise” and “comprises,” have correspondingly varied meanings. Thus, for example, a polynucleotide “comprising” a sequence encoding a protein may consist exclusively of that sequence or may include one or more additional sequences.

As used herein, the term “about” when used in reference to a recited numerical value includes the recited numerical value and numerical values within plus or minus ten percent of the recited value.

As used herein, the term “between” when used herein in reference to a range of numerical values encompasses the numerical values at each endpoint of the range. For example, a resolution of between 1 μm and 50 μm is inclusive of the values 1 μm and 50 μm.

As used herein, the term “substantially” means “approximately” and may be applied to modify any representation (quantitative or otherwise) that could permissibly vary without resulting in a change in the basic function to which it is related.

Any description of prior art documents herein, or statements herein derived from or based on those documents, is not an admission that the documents or derived statements are part of the common general knowledge of the relevant art.

For the purposes of description all documents referred to herein are incorporated by reference in their entirety unless otherwise stated.

### DETAILED DESCRIPTION

The present invention provides three-dimensional fiber constructs with components patterned at high resolution. The invention also provides methods for the assembly of such constructs facilitating the micropatterning of components (e.g. cells and/or biologics) at high resolution in a three dimensional environment. Although the fiber constructs of the invention are particularly beneficial in the field of tissue engineering it will be understood that no limitation exists regarding the application for which they are utilised.

In general, fiber constructs of the invention comprise a basic biostructural unit in the form of a sinusoid (also referred to hereinafter as a “secondary sinusoid structure” or a “secondary sinusoid unit”). Each secondary sinusoid unit comprises a plurality of regularly arranged fibers fused together at a very small distance such that components (e.g. cells) encapsulated within different fibers can be patterned at a high resolution in a three-dimensional environment. In some embodiments, fiber-assembled tissue constructs of the invention are composed of sinusoid units comprising a central fiber unit wrapped by additional fiber(s). Each fiber in the unit allows for the incorporation of specific components. Due to the small diameter of the fibers provided herein, their assembly into secondary sinusoid units allows three-dimensional micropatterning of components encapsulated in the fibers at a resolution of less than 50 μm. A number of currently available techniques for tissue engineering fail to provide this level of resolution (e.g. 3D cell plotting and organ printing) reducing their comparative effectiveness.

Furthermore, in comparison to techniques such as dielectrophoretic cell patterning and laser-guided direct writing, fiber constructs of the present invention provide a higher density of cells in a structurally stable three-dimensional construct. Laser-guided direct writing has also not been extended to robust three-dimensional structures which are important for clinically relevant constructs. Moreover, cell patterning is done in series and is thus time consuming. In contrast, the methods of producing tissue-assembled fiber constructs described herein are faster and more practical.

The present invention also provides for components that are spatially defined within a continuum (i.e. fiber matrix) within which the capacity for internal components (e.g. cells) of different component layers to migrate and interact is restricted. These fibers (hereinafter to be also referred to as “multi-component” fibers) allow for co-culture of different components (e.g. different cell types) in their respective niches within a single fiber, a feature which may be exploited to increase the resolution of three-dimensional micropatterning and achieve optimal fiber function. Furthermore, the incorporation of multi-component fibers into secondary sinusoid units provides a means of further increasing the resolution of encapsulated components as the resolution within each domain of the multi-component fiber can be less than 50 μm.

For example, encapsulation of components within separate domains of a cell-free multi-component fiber may facilitate three-dimensional micropatterning of the components within the fiber at a resolution of between about 1 μm and about 50 μm, less than about 40 μm, less than about 30 μm, less than about 20 μm, or less than about 10 μm.

Encapsulation of components within separate domains of a cell-laden multi-component fiber may facilitate three-dimensional micropatterning of the components within the fiber at a resolution of between about 1 μm and about 40 μm, less than about 40 μm, less than about 30 μm, less than about 20 μm, or less than about 15 μm.

Methods for the production of multi-component fibers are also provided herein.

The size and viability of engineered tissue are constrained by the availability and accessibility of vasculatures that provide efficient transportation networks for nutrients and waste. Adequate mass transfer of nutrients and waste can generally only occur over a thickness of ˜4-7 cell layers, or a maximum of 100-200 μm, which limits the thickness of viable tissue constructs. Tertiary constructs of the invention may comprise individual sinusoid units with a central fiber. Central fibers of sinusoid units may collectively provide a series of parallel channels having close proximity to the eventual vasculature. The basic sinusoid structure of the unit also increases the propensity for vascularisation. Once integrated into the host vasculature the channels provide efficient nutrient and/or waste transportation for neighbouring cells. Tissue fiber constructs of the invention thus provide a solution to the problem of mass transfer of nutrients and wastes faced by thicker tissue constructs.

### Secondary Sinusoid Units

The present invention provides fiber constructs in which individual components can be micropatterned at high resolution in a three-dimensional environment. Although fiber constructs of the invention are advantageous for tissue engineering, no particular limitation exists regarding the area of technology in which they may be utilised.

Fiber constructs of the invention include a secondary sinusoid unit comprising at least two primary fibers. A secondary sinusoid unit consists of two or more fibers arranged in a parallel or substantially parallel bundle. The secondary sinusoid unit may be derived by wrapping at least one fiber around at least one central fiber and fusing the fibers together, or alternatively by rotating at least two fibers about a central axis and fusing the fibers together. Methods for the assembly of these secondary sinusoid units and tertiary structures comprising the same are provided in the sections below entitled “Assembly of secondary sinusoid units” and “Assembly of tertiary constructs”. The secondary sinusoid units may be assembled to form three dimensional tertiary construct(s) comprising fused secondary sinusoid units.

Individual fibers (hereinafter also referred to as “primary” fibers) utilised for assembling secondary sinusoid units of the invention generally comprise a matrix encapsulating one or more desired components. The matrix is generally porous supporting the migration and/or self assembly of components encapsulated within it.

In general, the air-liquid interface of a nascent primary fiber provides a membrane-like structure or “skin” around the external surface of the fiber.

In certain embodiments fiber constructs of the invention are used in biological applications such as tissue engineering. In these embodiments, primary fibers preferably comprise a matrix capable of supporting cells and the fiber matrix is preferably biocompatible or substantially biocompatible (meaning that it is suitable for introduction into a mammalian host and is not substantially toxic).

Although any suitable matrix may be used, it is preferred that the matrix is polymer-based. Preferably, the matrix is aqueous.

In certain embodiments, the fiber matrix is a hydrogel. As used herein, the term “hydrogel” refers to a hydrophilic polymeric network capable of absorbing water without dissolving (i.e. a water insoluble, water-containing material).

Suitable hydrogels include macromolecular and polymeric materials into which water and other small molecules (e.g. biologics such as extracellular matrix proteins and drugs) can easily diffuse. Non-limiting examples include hydrogels prepared by cross-linking of both natural and synthetic hydrophilic polymers via ionic, covalent, and/or hydrophobic bonds introduced by chemical cross-linking agents and/or electromagnetic radiation (e.g. ultraviolet light). For example, suitable hydrogels include those prepared by cross-linking of poly(vinyl pyrrolidone); polysaccharides (e.g. hyaluronic acid, chondroitin sulfate, dextran, alginate, heparin or heparin sulfate); poly(vinyl alcohol); polyethers (e.g. polyakyleneoxides including poly(ethylene oxide), poly(ethylene glycol), poly(ethylene oxide)-co-(poly(propyleneoxide) block copolymers); or proteins (e.g. albumin, ovalbumin, gelatin, polyamino acids or collagen).

The hydrogel may exist in a variety of configurations, including, for example, sheets, particles, rods, beads, or irregular shapes.

The polymer matrix may be natural or synthetic. Specific examples of suitable hydrogels composed of synthetic polymers include polyhydroxy ethyl methacrylate, and chemically or physically cross-linked polyacrylamide, poly(N-vinyl pyrolidone), polyvinyl alcohol, polyethylene oxide, and hydrolysed polyacrylonitrile. Specific examples of suitable hydrogels composed of organic polymer hydrogels include covalent or ionically cross-linked polysaccharide-based hydrogels such as the polyvalent metal salts of alginate, pectin, heparin, carboxymethyl cellulose, hyaluronate and hydrogels from gellan, pullulan, chitin, chitosan, and xanthan.

Polycationic polymers that may be used in the generation of hydrogels include, but are not limited to, chitin, chitosan, poly(lysine), polyglutamic acid, polyornithine, polyethyleneimine; galactosylated compounds of chitin, collagen, chitosan and methylated collagen; natural and synthetic carbohydrates; polypeptide polymers having a net positive charge; or combinations thereof.

Polyanionic polymers that may be used in the generation of hydrogels include, but are not limited to, alginate, gellan, chondroitin sulphate, hyaluronic acid, fibrinogen; terpolymer consisting of methyl methacrylate, hydroxyethyl methacrylate and methacrylic acid; carboxymethylated, phosphorylated and/or sulfated derivatives, which include those of cellulose, chitin and chitosan; deoxyribonucleic acid (DNA), ribonucleic acid (RNA) and their derivatives; natural and synthetic carbohydrate; polypeptide polymers having a net negative charge; or combinations thereof.

The porosity of the fiber matrix (e.g. a hydrogel matrix) is generally of a size that allows the migration of components (e.g. cells, proteins, growth factors, nutrients, cellular wastes) through the matrix.

In certain embodiments, the pore size of the matrix is between about 1 nanometer and about 20 micrometers. In other embodiments, the pore size of the matrix is less than about 20, 19, 18, 17 16, 15, 14, 13, 12, 11, 10, 9, 8, 7, 6, 5, 4, 3, 2 or 1 micrometers. The pore size of the matrix may be between about 1 nanometer and 1000 nanometers (i.e. 1 micrometer), between about 10 nanometers and 1000 nanometers, between about 10 nonometers and about 500 nanometers, or between about 10 nanometers and about 100 nanometers.

Suitable fiber matrices can be prepared using methods known in the art. Specific methods for producing chitin-based hydrogels are provided in the Examples of the present specification. Alternatively, suitable hydrogels or precursors thereof may also be purchased from various commercial sources.

Primary fibers utilised for assembling secondary sinusoid structures generally comprise one or more encapsulated component(s).

The fibers may comprise encapsulated biological components, non-limiting examples of which include cells and biologics (e.g. proteins, hormones, angiogenic factors, growth factors, drugs and the like). As contemplated herein, an “encapsulated” component may migrate within the fiber and/or in some cases migrate out of the fiber.

Without limitation to a particular mechanism or mode of action, it is postulated that migration of certain components out of a given fiber (and into surrounding fiber(s)) is a characteristic that may develop over time as the fiber is bioresorbable. For example, in the case of encapsulated cells, it is thought that migration out of the fiber may be hindered by a skin layer formed initially by the arrangement of hydrophilic/hydrophobic regions at the water-air interface when the fiber is drawn. It is thought that this skin may eventually be resorbed to allow cell migration out of and between fibers.

In certain embodiments, the fibers comprise encapsulated biological components. For example, the fibers may comprise a specific type of cell, or, a mixture of different cell types. In general, cells encapsulated in a fiber of the invention may move within the fiber. Although migration of the cell out of the fiber (and into surrounding fiber(s)) may be possible, the capacity to do so will generally depend on factors such as the degree to which the fiber is degradable along with the specific encapsulated biological component and/or polyelectrolyte complex (e.g. pore size).

Additionally or alternatively, the fibers may comprise a specific encapsulated biologic, or, a mixture of different encapsulated biologics. Encapsulated biologics may be conjugated to the fiber material and thus may generally only diffuse out of the fiber once the fiber degrades. Alternatively, encapsulated biologics may not be conjugated to the fiber material and may, in certain circumstances, diffuse out of the fiber. The capacity and the rate at which an encapsulated biologic move out of a given fiber (and into surrounding fiber(s)) will generally depend on factor(s) such as the size of the biologic, the concentration gradient of the biologic in the surrounding environment/medium and the pore size of the matrix.

In certain embodiments, fibers may comprise encapsulated microorganisms.

In accordance with the invention, primary fibers of fiber-assembled tissue constructs comprise an acellular matrix. In general, the acellular matrix is capable of supporting the migration and/or self assembly of encapsulated biological components (e.g. cells and/or biologics) residing within it. The cells may be autologous, allogeneic or xenogeneic to an intended recipient of the fiber construct. Any given cell type(s) may be encapsulated in the fiber. In addition, it will be understood that different individual fibers within a secondary sinusoid unit of the invention need not comprise identical cell type(s).

Non-limiting examples of cell types that may be encapsulated in the primary fibers include embryonic stein cells, adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, ceruminpus gland cells, eccrine sweat gland cells, apocrine sweat gland cells, MpH gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing P cells, glucagon-producing a cells, somatostatin-producing DELTA cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair, cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialised for touch, primary sensory neurons specialised for temperature, primary neurons specialised for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fiber cells, melanocytes, retinal pigmented epithelial cells, iris pigment epithelial cells, oogonium, oocytes, spermatocytes, spermatogonium, ovarian follicle cells, Sertoli cells, and thymus epithelial cells, hepatocarcinoma, or combinations thereof, or cell lines derived therefrom.

As mentioned above, the secondary sinusoid structure of the invention may comprise at least one central fiber. Accordingly, any one or more of the cell type(s) referred to in the preceding paragraph may be encapsulated in at least one central fiber of the sinusoid structure. Preferred, cell types that may be encapsulated in the central fiber include, but are not limited to, endothelial cells, epithelial cells and neurons. Additionally or alternatively, any one or more of the cell type(s) referred to in the preceding paragraph may be encapsulated in one or more fiber(s) that surround the central fiber.

The number of cells encapsulated in a given fiber will generally depend on factors such as the length and diameter of the fiber along with the size and morphology of the cells utilised, cell density, size of polyelectrolyte solution droplet, solution concentration, draw rate, and type of polyelectrolyte. Preferably, primary fibers comprise a high density of cells, although the density of cells will depend on the particular application.

In certain embodiments, the fiber comprises a cell density of between about 50 million and 200 million cells/ml. Preferably, the fiber comprises a cell density of between about 100 million and 200 million cells/ml, and more preferably between about 100 million and 150 million cells/ml.

In addition to encapsulated cells, fiber-assembled tissue constructs of the invention may comprise other additional components. The additional components may be encapsulated in an additional encapsulant (e.g. microspheres, micelles).

Non-limiting examples of additional component(s) include proteins (e.g. extracellular matrix proteins such as collagen, elastin, pikachurin; cytoskeletal proteins such as actin, keratin, myosin, tubulin, spectrin; plasma proteins such as serum albumin; cell adhesion proteins such as cadherin, integrin, selectin, NCAM; and enzymes), hormones or growth factors (e.g. insulin, insulin-like growth factor, epidermal growth factor, oxytocin); neurotransmitters (e.g. serotonin, dopamine, epinephrine, norepinephrine, acetylcholine); angiogenic factors (e.g. angiopoietins, fibroblast growth factor, vascular endothelial growth factor, matrix metalloproteinase enzymes); amino acids; galactose ligands; nucleic acids (e.g. DNA, RNA); drugs (e.g.

The additional components may be obtained from any source (e.g. humans, other animals, microorganisms). For example, they may be produced by recombinant means or may be extracted and purified directly from a living source. It is also contemplated that different encapsulated cell types within fiber-assembled tissue constructs of the invention may provide a source of the additional components. In certain embodiments, fiber-assembled tissue constructs of the invention comprise encapsulated biologics.

Primary fibers utilised for assembling secondary sinusoid units of the invention may or may not comprise cells.

Without imposing any particular restriction or limitation, the diameter of a primary fiber comprising cells may be between about 1 micrometer and about 200 micrometers, between about 2 micrometers and about 200 micrometers, between about 2 micrometers and about 100 micrometers, or between about 2 micrometers and about 50 micrometers.

In general, and again without imposing any particular restriction or limitation, the diameter of a cell-free fiber may be between about 1 micrometer and about 500 micrometers, between about 5 micrometers and about 500 micrometers, between about 10 micrometers and about 500 micrometers, between about 10 micrometers and about 400 micrometers, between about 10 micrometers and about 300 micrometers, between about 10 micrometers and about 200 micrometers, between about 10 micrometers and about 100 micrometers, or between about 10 micrometers and about 80 micrometers.

Without imposing any particular restriction or limitation, the length of a primary fiber will generally be in the range of about 0.1 cm to about 50 cm. For example, the length may be between about 1 cm and about 50 cm, between about 10 cm and about 50 cm, between about 10 cm and about 40 cm, or between about 1 cm and about 40 cm. In one embodiment, the length of the fiber is greater than about 10 cm.

In general, a secondary sinusoid unit in accordance with the invention comprises a plurality of primary fibers assembled to form a higher order structure. A secondary sinusoid unit may comprise a periodic pattern which varies positively and/or negatively symmetrically about an axis. It will be recognized a secondary sinusoid unit may not be a perfect sinusoid, but also an approximation of a sinusoid. A secondary sinusoid structure may resemble, for example, a hepatic sinusoid, a spleen sinusoid or a sinusoid of the bone marrow. The secondary sinusoid units of the invention are assembled by combining two or more primary fibers. Accordingly, the secondary sinusoid unit may comprise two, three, four, five, six, seven, eight, nine, ten or more than ten primary fibers.

A secondary sinusoid unit comprising two primary fibers may comprise a single central fiber wrapped by a single “outer” primary fiber. Alternatively, a secondary sinusoid unit comprising two primary fibers may not comprise a central primary fiber and be formed by rotating the two fibers about an axis.

A secondary sinusoid unit comprising three or more primary fibers may comprise one or more central primary fibers wrapped by one or more outer primary fibers. Alternatively, a secondary sinusoid unit comprising three or more primary fibers may not comprise a central primary fiber and be formed by rotating the fibers about an axis.

The secondary sinusoid units of the invention may be assembled into tertiary structures comprising two or more secondary sinusoid units. Tertiary structures of the invention therefore comprise a plurality of repeating secondary sinusoid units. In general, it is preferable that repeated secondary sinusoid units are arranged in such a way that the vertical axis of each unit is parallel or substantially parallel. This minimises the distance between repeating units thus increasing resolution and facilitating closer communication between different individual sinusoid units.

The assembly of multiple different secondary sinusoid units into tertiary structures allows the micropatterning of components in primary fibers (e.g. cells and/or biologics) at high resolution in a three-dimensional environment. For example, co-encapsulation of specific cells and biologics within separate secondary sinusoid units allows the creation of separate three-dimensional niche environments for the growth and/or differentiation of different cell types facilitated by the localisation of specific chemical and/or biological cues in within secondary sinusoid units.

In general, cell patterning resolution in three-dimensional tissue-assembled fiber constructs of the invention is less than about 100 μm, preferably less than about 75 μm, more preferably less than about 50 μm, still more preferably less than about 40 μm, and even still more preferably less than about 30 μm. Accordingly, the invention provides structurally stable three-dimensional tissue-assembled fiber constructs with a high density of cells (e.g. between about 50 million and 200 million cells/ml) at high cell patterning resolution.

The arrangement of primary fibers into secondary sinusoid units in accordance with the invention facilitates localised communication between components in different primary fibers. For example, cells present in separate primary fibers of secondary sinusoid units are able to communicate with each other via the release of factors capable of migrating through the matrix of each fiber. Accordingly, the subsequent assembly of secondary sinusoid units into tertiary structures allows the micropatteming of components within fibers (e.g. cells) at high resolution within a three-dimensional environment. A fiber-assembled tissue construct of the invention may be constructed in such a way to direct the simultaneous differentiation and/or growth of different cell types in the same three-dimensional environment by localisation of chemical and/or biological cues (i.e. non-encapsulated components capable of migrating within and between fibers) within different secondary sinusoid unit(s).

Engineered tissues generally lack an initial blood supply making it difficult for the implanted cells to obtain sufficient nutrients to survive, and/or function efficiently. Furthermore, the lack of initial vascularisation makes the expulsion of cellular waste materials problematic resulting in the build up of toxins and other undesirable compounds.

In certain embodiments of the invention fiber-assembled tissue constructs comprise secondary sinusoid unit(s) comprising one or more “outer” fibers wrapped around one or more central fibers. The structure of the secondary sinusoid unit facilitates cell-cell communication between cells in outer fiber(s) with cells in the central fiber(s). In the case of a central fiber comprising cell types capable forming blood vessels (e.g. endothelial cells), the sinusoid structure may facilitate the formation of a network of parallel channels of blood vessels in tertiary constructs of the invention that provide sufficient nutrient and waste transportation for neighbouring cells once integrated into the host vasculature. In addition, the presence of endothelial cells in primary fibers may influence the development and function of adjacent cells (e.g. cells in adjacent fibers), such as cardiomyocytes, hepatocytes, pancreatic cells, thyroid cells and hematopoietic stem cells.

The spatially and quantitatively defined cell-cell interactions afforded by the secondary sinusoid units of the invention are therefore useful in the construction of tissue-engineered implants of higher viability and functionality.

### Multiple-Component Fibers

The invention provides primary multiple-component fibers (also referred to hereinafter as “multi-component fiber(s)”) comprising components that are spatially defined within a continuum. The provision of multi-component fibers allows the compartmentalisation of specific components within a single fiber. Individual components within a given multi-component fiber of the invention (e.g. different cell types) may be encapsulated in distinct layers (also referred to hereinafter as “domains”) thus allowing the micropatterning of cells within the individual fiber.

A multi-component fiber of the invention comprises at least two domains. Although no particular restriction exists regarding the number of domains, multi-component fibers may comprise between,two and ten domains, preferably between two and five domains, and more preferably two or three domains.

In general, the diameter of a multi-component fiber of the invention will be influenced by the number of individual domains within it. Each domain of a multi-component fiber arises from a nascent fiber drawn from one interface. These domains may be homogeneous or heterogeneous depending on the composition of the solutions used to draw the fiber.

In general, the air-liquid interface of a nascent multi-component fiber provides a membrane-like structure or “skin” around the external surface of the multi-component fiber. The matrix at the “barrier” of different internal domains within the multi-component fiber is generally a continuum, each domain comprising nuclear fibers which surround encapsulated components (e.g. cells). As the nuclear fibers are in parallel (or substantially in parallel), the domains are distinctly separated at the beginning. Components are generally able to move within the space between nuclear fibers and may thus eventually cross an internal “barrier”. Encapsulated components (e.g. biologics) which are conjugated to the nuclear fibers generally remain in separate domains. Other non-conjugated components may move across domains within the multi-component fiber (e.g. by diffusion).

Multi-component fibers of the invention may measure tens to hundreds of micrometers (μm) in diameter. Multi-component fibers utilised for assembling secondary sinusoid units of the invention may or may not comprise cells. Without imposing any particular restriction or limitation, the diameter of a multi-component fiber comprising cells may be between about 1 μm and about 500 μm, between about 1 μm and about 200 μm, between about 1 μm and about 100 μm, or between about 1 μm and about 50 μm. In general, and again without imposing any particular restriction or limitation, the diameter of cell-free multi-component fiber may be between about 1 μm and about 500 μm, between about 5 μm and about 400 μm, between about 5 μm and about 300 μm, between about 5 μm and about 200 μm, between about 5 μm and about 100 μm, or between about 5 μm and about 50 μm.

The incorporation of multi-component fiber(s) into secondary sinusoid units provides a means of increasing resolution as components can be micropatterned at high resolution within a given multi-component fiber.

For example, encapsulation of components within separate domains of a cell-free multi-component fiber may facilitate three-dimensional micropafterning of the components within the fiber at a resolution of between about 1 μm and about 50 μm, less than about 40 μm, less than about 30 μm, less than about 20 μm, or less than about 10 μm.

Encapsulation of components within separate domains of a cell-laden multi-component fiber may facilitate three-dimensional micropatterning of the components within the fiber at a resolution of between about 1 μm and about 40 μm, less than about 40 μm, less than about 30 μm, less than about 20 μm, or less than about 15 μm.

Without imposing any particular restriction or limitation, the length of a multi-component fiber will generally be in the range of about 0.1 cm to about 50 cm. For example, the length may be between about 1 cm and about 50 cm, between about 10 cm and about 50 cm, between about 10 cm and about 40 cm, or between about 1 cm and about 40 cm. In one embodiment, the length of the fiber is greater than about 10 cm.

Although it is contemplated that a multi-component fiber of the invention may be used as a separate entity for applications such as tissue engineering, it will also be understood that they may be used in the assembly of secondary sinusoid units.

Accordingly, a secondary sinusoid unit of the invention may be assembled from multi-component fibers (only) or a combination of basic fibres and multi-component fibers. Multi-component fibers may form central fibre(s) of a secondary sinusoid unit and/or outer fibre(s) surrounding central fibre(s).

Any number of multi-component fibers may be incorporated into a secondary sinusoid unit of the invention. Accordingly, a secondary sinusoid unit may comprise two, three, four, five, six, seven, eight, nine, ten or more than ten multi-component fibers.

In general, multi-component fibers of the invention comprise a matrix. The matrix is generally porous supporting the migration and/or self assembly of components encapsulated within it. Preferably, the matrix is a hydrogel. Non-limiting examples of suitable matrix materials and including polymers and hydrogels methods for their generation are provided in the section above entitled “Sinusoid Biostructural Units”.

The porosity of the fiber matrix (e.g. a hydrogel matrix) is generally of a size that allows the migration of components (e.g. cells, proteins, growth factors, nutrients, cellular wastes) within separate domains of a multi-component fiber and/or between different domains within a multi-component fiber and/or in/out of the multi-component fiber. In certain embodiments, the pore size of the matrix is between about 1 nanometer and about 20 micrometers. In other embodiments, the pore size of the matrix is less than about 20, 19, 18, 17 16, 15, 14, 13, 12, 11, 10, 9; 8, 7, 6, 5, 4, 3, 2 or 1 micrometers. The pore size of the matrix may be between about 1 nanometer and 1000 nanometers (i.e. 1 micrometer), between about 10 nanometers and 1000 nanometers, between about 10 nonometers and about 500 nanometers, or between about 10 nanometers and about 100 nanometers.

Multi-component fibers generally comprise one or more constituent(s). Typically, the constituents are biological and/or chemical constituents. For example, multi-component fibers may comprise one or more different types of cells. Accordingly, individual components of multi-component fibers may comprise cells encapsulated in an acellular scaffold matrix. Any given cell type(s) may be encapsulated in one or more domains of a multi-component fiber. Although different cells types are typically compartmentalised in different domains of the fiber, it is also contemplated that individual domain(s) of the fiber may comprise a mixture of cell types. Cells encapsulated in multi-component fibers may be autologous, allogeneic or xenogeneic to an intended recipient of the fiber.

Non-limiting examples of cell types that may be encapsulated in the multi-component fibers include those which are suitable for encapsulation in the primary fibers of the invention as provided in the section above entitled “Sinusoid Biostructural Units”.

In addition to encapsulated cells, multi-component fibres of the invention may comprise other additional constituents. The additional constituents may be encapsulated or non-encapsulated. Non-limiting examples of the additional constituents(s) and sources of the same include those described for inclusion in primary fibers of the invention as provided in the section above entitled “Sinusoid Biostructural Units ”.

In general, the acellular matrix is capable of supporting the migration of constituents and/or the self assembly of encapsulated cells residing within it. It will be understood that cells of one domain may interact with cells of another adjacent component, and further that cells may migrate between individual domains of the fiber.

### Fiber-Assembled Constructs

The invention provides methods for the assembly of primary fibers and multi-component fibers. Also provided are methods for the assembly of primary fibers and/or multi-component fibers into secondary sinusoid units of the invention. Secondary sinusoid units may be further assembled into tertiary constructs.

### Assembly of Multiple Component Fibers

Certain embodiments of the invention relate to methods for patterning cells or other materials within an individual fiber. Accordingly, the invention provides methods for the assembly of multi-component fibers in which components are spatially defined within a continuum.

Multi-component fibers of the invention are generally assembled by multi-interfacial polyelectrolyte complexation (MIPC).

By way of example, in certain embodiments of the invention a two-component fiber may be formed by dispensing droplets of three polyelectrolyte solutions onto a suitable surface such that the droplets faun a substantially linear arrangement comprising a central droplet flanked by two outer droplets. The arrangement is such that the central solution has an opposite charge to each outer solution. For example, the central droplet may be a solution comprising a suitable polycationic polymer (e.g. chitin, chitosan, poly(lysine), polyglutamic acid, polyornithine, polyethyleneimine; galactosylated compounds of chitin, collagen, chitosan and methylated collagen; natural and synthetic carbohydrates; polypeptide polymers having a net positive charge; or combinations thereof) while each of the outer droplets may be a solution comprising a suitable polyanionic polymer (e.g. alginate, gellan, chondroitin sulphate, hyaluronic acid, fibrinogen; terpolymer consisting of methyl methacrylate, hydroxyethyl methacrylate and methacrylic acid; carboxymethylated, phosphorylated and/or sulfated derivatives, which include those of cellulose, chitin and chitosan; deoxyribonucleic acid (DNA), ribonucleic acid (RNA) and their derivatives; natural and synthetic carbohydrate; polypeptide polymers having a net negative charge; or combinations thereof).

Depending on the intended application of the multi-component fiber, the polyanionic and/or polycationic solutions may comprise components such as, for example, cells, biologics, proteins, hormones, angiogenic factors, growth factors, drugs and the like.

Opposing surfaces of each adjacent solution are each in contact with the pointed tip of an appropriate elongated instrument (e.g. a pipette tip) preventing or substantially preventing contact between the central droplet and each adjacent outer droplet. Accordingly the three individual droplets are separated by the two pointed tips positioned on either side of the central droplet in a substantially linear arrangement. Alternatively, the droplets may already be in contact, with each adjacent pair forming a stable interface, prior to drawing the tip upwards.

Preferably, each pointed tip in contact with the solutions is coated with an adhesive to allow adherence to fibres drawn from the polyelectrolyte solutions. In general, the adhesive may be any material capable of maintaining contact (directly or indirectly) between the tip and each polyelectrolyte solution. Any suitable adhesive may be used, including organic and inorganic materials. The organic materials may be polymeric compounds.

Non-limiting examples of organic adhesives that may be used in the methods of the present invention include fibrin glue, polyvinylpyrolidone, polyvinylpyrolidone/vinyl acetate copolymers, cyanoacrylate gel, platelated gel, chitosan or gelatin-resorcin-formaldehyde (GRFG). Additional non-limiting examples include organic polymeric compositions represented by the group of alkyd resins, polyvinyl acetaldehydes, polyvinyl alcohols, polyvinyl acetates, poly(ethylene oxide), polyacrylates, ketone resins, polyvinylpyrolidone, polyvinylpyrolidone/vinyl acetate copolymer, polyethylene glycols of 200 to 1000 molecular weight and polyoxyethylene/polyoxopropylene block copolymers (Polyox), silicone resins and silicone based pressure sensitive adhesives. Pressure sensitive adhesives are well known in the art and commercially available (e.g. those available from Dow Coming Company under the trade designation BIO-PSA).

Each tip is drawn upwards at an appropriate rate (e.g. between about 0.05 mm and 0.5 mm/second and preferably about 0.3 mm/second) maintaining the contact between the tips and droplets and bringing opposing surfaces of the droplets into contact resulting in the formation of two stable interfaces. Alternatively, the droplets may already be in contact, with each adjacent pair fanning a stable interface, prior to drawing the tip upwards at an appropriate rate (e.g. between about 0.05 mm and 0.5 mm/second and preferably about 0.3 mm/second). Each interface exists at the region of contact between the oppositely charged central and outer droplets. A nascent fibre forms from each interface and continued upward drawing culminates in the fusion of the nascent fibers resulting in a single two-component fiber. Preferably, drawing of fibres is conducted in a humid atmosphere to protect cells and other constituents within the fibers from drying.

Multi-component fibers comprising more than two domains may be formed using an extension of the method described above. For example, a four-component fiber may be produced using the same technique but extending the (substantially) linear arrangement of (opposing) oppositely charged polyelectrolyte solutions to five. The additional solutions may comprise components (e.g. cells, biologics and the like) if desired. The five solutions are separated from each other by virtue of four separate tips. Each tip is drawn upwards at an appropriate rate (e.g. between about 0.05 mm and 0.5 mm/second and preferably about 0.3 mm/second) maintaining the contact between the tips and droplets and bringing opposing surfaces of the droplets into contact resulting in the formation of four stable interfaces. Alternatively, the droplets may already be in contact, with each adjacent pair forming a stable interface, prior to drawing the tip upwards at an appropriate rate (e.g. between about 0.05 mm and 0.5 mm/second and preferably about 0.3 mm/second). A nascent fiber forms from each interface and continued upward drawing culminates in the fusion of the nascent fibers resulting in a single four-component fiber.

The skilled addressee will readily recognise that the formation of multi-component fibres with more than three domains can be achieved by incorporating additional polyelectrolyte solutions and tips and performing the process described above.

Without being limited to a particular mechanism or mode of action, it is postulated that assembly of multi-component fibers using the methods of the invention involves the process of nuclear fiber formation and coalescence. The method of fiber formation by IPC is postulated to occur via a multistep mechanism in which the first step involves the formation of a polyelectrolyte complex film at the interface between two oppositely charged polyelectrolytes, which constitutes a viscous barrier that prevents bulk mixing of the two polyelectrolytes. When the interface is drawn upwards by a vertical motion, the polyelectrolyte film is broken into separate domains which may nucleate further complex formation by consuming polyelectrolytes from the surrounding solution, forming submicron nuclear fibers. These nuclear fibers are then thought to coalesce to form the primary fiber and beads spaced out at regular intervals along the fiber axis.

The fusion of nascent fibers from two or more interfaces, if occurring at the point where the nascent fibers leave the solution-air interface is believed to lead to multiple sets of nuclear fibers, clearly defined in space, within the same primary fiber.

The successful assembly of multi-component fibers using the methods of the invention necessitated overcoming the technical difficulty of forming two interfaces close enough to allow fusion of nascent fiber at the solution-air interface. That problem was addressed by devising the configuration of polyelectrolyte solutions and tips described in the preceding paragraphs, with a polycation droplet intervening between two polyanion droplets and forming interfacial complexes. As nascent fibers are drawn from the interfaces they come gradually closer to each other due to depletion of the polycation and drifting of the interface or manual action facilitating the eventual fusion event and forming a multi-component fiber.

### Assembly of Secondary Sinusoid Units

Certain embodiments of the invention relate to methods for the assembly of secondary sinusoid units described herein.

By way of example, secondary sinusoid units of the invention comprising a central fiber wrapped by a plurality of outer fibers may be prepared by the following method.

Pairs of oppositely charged polyelectrolyte solutions are dispensed on the surface of a suitable support in a pre-determined pattern. Oppositely charged pairs are positioned closely together but are not in contact with each other. Preferably the support is circular in shape although this is not essential. The arrangement is such that a pair of solutions intended to form the central fiber is dispensed towards the centre of the support and most preferably at the centre of the support. Pairs of oppositely charged polyelectrolyte solutions intended to form outer fibers in the sinusoid unit are dispensed toward the perimeter of the support. In preferred embodiments, individual pairs of oppositely charged polyelectrolyte solutions intended to form outer fibers are equidistant or substantially equidistant from the central pair of solutions. Moreover, it is also preferred that individual pairs of oppositely charged polyelectrolyte solutions intended to form outer fibers are dispensed such that are evenly spaced around a circumference of the central pair of solutions.

Pairs of oppositely charged polyelectrolyte solutions may comprise a polycationic polymer solution as one component of the pair (e.g. chitin, chitosan, poly(lysine), polyglutamic acid, polyornithine, polyethyleneimine; galactosylated compounds of chitin, collagen, chitosan and methylated collagen; natural and synthetic carbohydrates; polypeptide polymers having a net positive charge; or combinations thereof) and a polyanionic polymer solution as the other component of the pair (e.g. alginate, gellan, chondroitin sulphate, hyaluronic acid, fibrinogen; terpolymer consisting of methyl methacrylate, hydroxyethyl methacrylate and methacrylic acid; carboxymethylated, phosphorylated and/or sulfated derivatives, which include those of cellulose, chitin and chitosan; deoxyribonucleic acid (DNA), ribonucleic acid (RNA) and their derivatives; natural and synthetic carbohydrates; polypeptide polymers having a net negative charge; or combinations thereof).

Depending on the intended application of the sinusoid unit, the polyanionic and/or polycationic solutions may comprise constituents such as, for example, cells, biologics, proteins, hormones, angiogenic factors, growth factors, drugs and the like, non-limiting examples of which are provided in the section above entitled “Sinusoid Biostructural units”.

Fibers may be drawn from each pair of oppositely charged polyelectrolyte solutions on the support, preferably in tandem, by applying a pointed tip (e.g. a pipette tip) coated with an adhesive such that opposing sides of the tip contact opposing surfaces of each adjacent solution (solutions will generally be applied to the support in the form of individual droplets). Preferably, each pointed tip in contact with the droplets is coated with an adhesive to allow adherence to fibres drawn from the polyelectrolyte solutions.

Each tip is drawn upwards (preferably in parallel) at an appropriate rate (e.g. between about 0.05 mm and 0.5 mm/second and preferably about 0.3 mm/second) maintaining the contact between the tips and bringing opposing surfaces of the droplets into contact to form stable interfaces. Alternatively, the droplets may already be in contact, with each adjacent pair forming a stable interface, prior to drawing the tip upwards at an appropriate rate (e.g. between about 0.05 mm and 0.5 min/second and preferably about 0.3 mm/second). Continued upward drawing motion results in the formation of nascent elongated fibers. Preferably, drawing of fibres is conducted in a humid atmosphere to protect cells and other components within the fibers from drying. Upon reaching the desired height/thickness, outer fibers may be rotated about a central axis formed by the central fiber allowing outer fibers to surround the central fiber and eventually causing all fibers (i.e. both central and outer fibers) to meet at a point. Fibers may then be fused by application of a suitable reagent (e.g. any of the polycationic and polyanionic polymers referred to in this subsection (see preceding paragraphs above), multivalent cations and anions such as calcium ions (Ca²⁺) and iron ions (Fe³⁺), and sodium alginate) to the fiber fusion point followed by continued upward drawing allowing the droplet to travel down the nascent construct and fuse the fibers via secondary complexation resulting in the formation of a secondary sinusoid unit of the invention.

It will be recognised that variations of the technique described above may be used to modify the structure of the resulting sinusoid unit. For example, sinusoid units may be formed by performing the method without a central fiber (i.e. outer fibers only). In general, when a central fiber is generated using this technique the minimum number of outer fibers is two.

It will also be understood that one or more multi-component fibers may be incorporated into the structure of a secondary sinusoid units of the invention by replacing any number of pair(s) of oppositely charged polyelectrolyte solutions at any given position on the template with a sequential series of oppositely charged polyelectrolyte solutions (numbering three or more separate droplets) and modifying the process to draw the multi-component fiber(s) as described in the subsection above entitled “Assembly of multiple component fibers”. A secondary sinusoid unit of the invention may comprise a mixture of multi-component fibers and primary fibers, multi-component fibers only, or primary fibers only. The multi-component fiber(s) may form central and/or outer fibers of a secondary sinusoid unit. The multi-component fiber(s) may comprise any number of components (i.e. compartments) within the fiber. Any one or more components of the multi-component fiber(s) may comprise constituents such as, for example, cells, biologics, proteins, hormones, angiogenic factors, growth factors, drugs and the like, non-limiting examples of which are provided in the section above entitled “Sinusoid Biostructural units”.

In alternative embodiments, secondary sinusoid units of the invention are formed by a variation of the method described above. Nascent fibers are drawn from oppositely charged polyelectrolyte solutions in a similar or identical manner but instead of being fused by a reagent such as sodium alginate they are subjected to continuous rotation which twists nascent fibers to form a rope-like structure. The fibers are fused together due to secondary complexation resulting from the compression generated by the twisting motion. The speed at which a given fiber may be twisted (i.e. rotated) around one or more other fibers may be between about 1 rpm and about 750 rpm, between about 1 rpm and about 600 rpm, between about 1 rpm and about 500 rpm, between about 1 rpm and about 400 rpm, between about 1 rpm and about 300 rpm, between about 1 rpm and about 200 rpm, or between about 1 rpm and about 100 rpm. This method of assembling secondary sinusoid units of the invention can be used with or without a centre fiber. In general, when a central fiber is generated using this technique the minimum number of outer fibers is one. It will also that be recognised that one or more multi-component fibers may be incorporated into the structure of secondary sinusoid units of the invention generated by this technique by modifying the process to draw multi-component fiber(s) (see section above entitled “Assembly of multiple component fibers”). A basic or multi-component fiber utilised to form a secondary sinusoid unit by this method may comprise may comprise constituents such as, for example, cells, biologics, proteins, hormones, angiogenic factors, growth factors, drugs and the like, non-limiting examples of which are provided in the section above entitled “Sinusoid Biostructural units”.

### Tertiary Constructs

The secondary sinusoid units of the invention may be assembled into tertiary structures comprising two or more secondary sinusoid units. Tertiary structures of the invention therefore comprise a plurality of repeating secondary sinusoid units. In general, it is preferable that repeated secondary sinusoid units are arranged in such a way that the vertical axis of each unit is parallel or substantially parallel. This minimises the distance between repeating units thus increasing resolution and facilitating closer communication between different individual sinusoid units.

The assembly of multiple different secondary sinusoid units into tertiary structures allows the micropatterning of components in primary fibers (e.g. cells and/or biologics) at high resolution in a three-dimensional environment. For example, co-encapsulation of specific cells and biologics within separate secondary sinusoid units allows the creation of separate three-dimensional niche environments for the growth and/or differentiation of different cell types facilitated by the localisation of specific chemical and/or biological cues in within secondary sinusoid units.

In general, cell patterning resolution in three-dimensional tissue-assembled fiber constructs of the invention is less than about 100 μm, preferably less than about 75 μm, more preferably less than about 50 μm, still more preferably less than about 40 μm, and even still more preferably less than about 30 μm. Accordingly, the invention provides structurally stable three-dimensional tissue-assembled fiber constructs with a high density of cells (e.g. between about 50 million and 200 million cells/ml, between about 100 million and 200 million cells/ml, and more preferably between about 100 million and 150 million cells/ml) at high cell patterning resolution.

In general, tertiary structures may be formed by fusing layers of secondary sinusoid units. It is preferred that when fused together, individual sinusoid units are arranged in such a way that the vertical axis of each unit is parallel or substantially parallel. This may be achieved, for example, by spooling nascent sinusoid units generated by the methods of the invention (see subsection above entitled “Assembly of secondary sinusoid units”) and fusing them together in layers.

Fibers of sinusoid units may be fused to form tertiary structures using any suitable reagent, by application of a suitable reagent (e.g. any of the polycationic and polyanionic polymers referred to in this subsection (see preceding paragraphs above), multivalent cations and anions such as calcium ions (Ca²⁺) and iron ions (Fe³⁺), and sodium alginate).

By way of example only, tertiary structures may be assembled by spooling sinusoid units produced in accordance with the methods provided herein on a two-pronged collection rod, fusing the individual sinusoids by dipping into solutions containing polyelectrolytes (e.g. chitosan, WSC, RMC and alginate) and/or multivalent ions (e.g. Ca²⁺, Fe³⁺). The assembled tertiary structures may then be removed from the collecting rod by cutting them at both ends (e.g. using a scalpel). Tertiary constructs may then be stored, for example, in an appropriate culture medium.

### Apparatus for Assembly

The invention provides an apparatus suitable for drawing nascent fibers (primary fibers, multi-component fibers and combinations thereof) from polyelectrolyte solutions and assembling them into secondary sinusoid units of the invention.

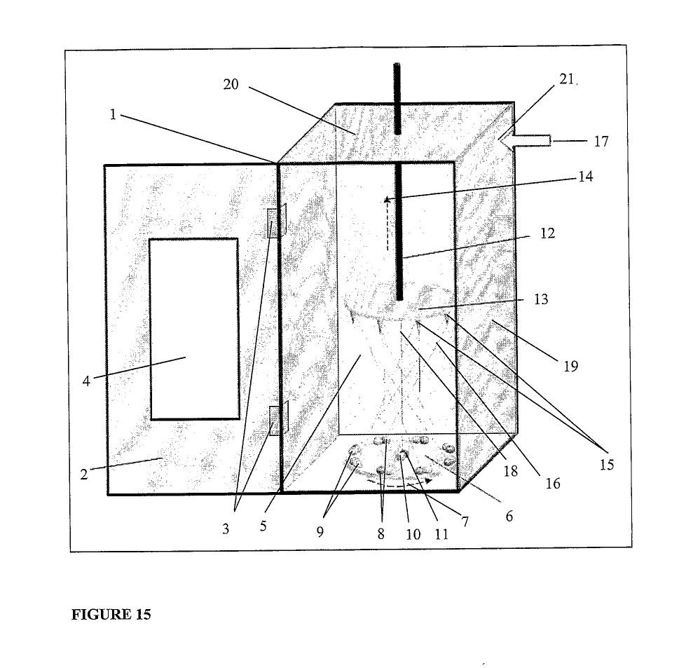

Turning to FIG. 15, the apparatus comprises a rotating template (6) capable of rotation in at least one direction (7). A fiber drawing template (13) is positioned above the rotating template, each template sharing a common central vertical axis defined by a linear motor shaft (12). The downward facing surface of the fiber drawing template has a series of downward facing protruding tips (15) around the full circumference of its edge and a single downward facing protruding tip at its centre. The tips are coated with adhesives facilitating adhesion to fibers (16, 18).The linear motor shaft (12) is attached to the upper surface of the fiber template. The fiber drawing and rotating templates are both housed in a rectangular box (1) having elongate vertical sides (19) and a top (20) firmly supporting the linear motor shaft in a vertical position. The box has a hinged (3) door (2) with a transparent section (4) allowing a user to view its interior (5) when the door is closed. The box has a flow inlet (21) allowing the flow of water vapour into the interior of the box. The inlet directs the flow of water vapour (17) into interior of the box in such a way that the flow is circulated within the box interior but not directed at fibers (16, 18).

In use, paired droplets of oppositely charged polycationic (9, 10) and polyanionic polymer solutions (8, 11) are deposited in a pre-determined pattern on the upper surface of the rotating template (6). Members of individual pairs may or may not be in direct contact. The linear motor shaft (12) which is driven by a motor is then used to lower the fiber drawing template (13) in a downward direction towards the upper surface of the rotating template until individual downward facing protruding tips (15) insert between and paired droplets of oppositely charged polycationic (9, 10) and polyanionic polymer solutions (8, 11), coming into and maintaining contact with each droplet of the pair. The linear motor shaft is then used to draw the fiber drawing template and protruding tips upwardly at an appropriate rate. When members of individual pairs are not in direct contact prior to drawing, initial upward movement of the protruding tips brings opposing surfaces of droplets in each pair into contact. Alternatively, members of individual pairs may already be in contact prior to contact with the protruding tips, with each adjacent pair forming a stable interface, prior to drawing the tip upwards. Continued upward drawing motion results in the formation of nascent elongated fibers. Upon obtaining desired fiber length by continued upward movement of the fiber drawing template and protruding tips, upward movement of the fiber drawing template is ceased and it is held in place by the linear motor shaft. This leaves a series of nascent fibers each of which has one end attached to the rotating template and the other end attached to a protruding tip of the fiber drawing template. Unidirectional rotation of the rotating plate about its vertical central axis relative to the fixed fiber drawing template allows the outer fibers (16) to wrap around the central fiber (18) and continued rotation causes the fibers to meet at a fiber fusion point located on the central fiber. A fusing reagent droplet is then applied to the fiber fusion point and the linear motor shaft then used to facilitate movement of the fiber drawing template allowing the droplet to travel down the nascent fiber and fuse the fibers via secondary complexation.

In order to obtain tertiary structures, the sinusoid fiber is removed from between the templates (e.g. by cutting with a scalpel at both ends) and spooled on a two-pronged collection rod. Individual sinusoids are then fused by dipping into solutions containing polyelectrolytes (e.g. chitosan, WSC, RMC and alginate) and/or multivalent ions (e.g. Ca²⁺, Fe³⁺). The assembled tertiary structures were then removed from the collecting rod by cutting them with a scalpel at both ends.

### Tissue Engineering

The present invention provides fiber constructs and methods for their assembly in which individual components can be micropatterned at high resolution in a three-dimensional environment. Although fiber constructs of the invention are advantageous for tissue engineering, no particular limitation exists regarding the area of technology in which they may be utilised.

Non-limiting examples of specific applications in the field of tissue engineering are provided below.

The fiber assembly technique presented here offers the potential of micro-patterning biological entities such as cells and/or biologics (ECM proteins, drugs) at high resolution in a three dimensional construct. This facilitates the creation of a three-dimensional niche environments for different cell type(s) by co-encapsulation of different cells and biologics within the same fiber or fiber construct. In this way, a fiber-assembled tissue construct of the invention can potentially be used to direct simultaneous differentiation of stem cells into different fates in the same three-dimensional environment by localization of chemical and biological cues within different primary biostructural units. Apart from creating niche microenvironments, the inherent structure of the secondary sinusoid unit facilitates cell-cell communication between cells in outer fiber(s) with cells in a central fiber. In the case of a central fiber containing endothelial cells, this inherent characteristic can potentially fill tertiary constructs with parallel channels of blood vessels that provide sufficient nutrient and waste transportation for neighbouring cells once these channels are integrated with the host vasculature. Besides functioning as conduits for mass transport of nutrients and oxygen, the endothelium has also been reported to influence the development and function of adjacent cells, such as cardiomyocytes, hepatocytes, pancreatic cells, thyroid cells and hematopoietic stem cells. Spatially and quantitatively defined cell-cell interactions as offered by the tissue constructs described herein are therefore useful in the construction of tissue-engineered implants of higher viability and functionality.

In certain embodiments fiber-assembled tissue constructs of the invention are assembled from secondary sinusoid units providing encapsulated endothelial cells in a central fiber and hepatocytes in outer fibers (the central and outer fibers being arranged in a sinusoid structure). In this way, a user of the tissue construct may co-culture the hepatocytes with the endothelial cells within the same construct in a three-dimensional microenvironment. The microenvironment may also be tailored to maintain hepatocyte function, for example, by incorporating galactose ligands in the form of galactosylated chitin in the fibers. The aligned central fibers containing endothelial cells may additionally comprise angiogenic factors for inducing in vivo angiogenesis within the construct.

Tubule formation is fundamental to the organization of epithelial cells in organs such as lung, kidney and the reproductive tracts. Accordingly, in other embodiments fiber-assembled tissue constructs of the invention are assembled from secondary sinusoid units providing encapsulated epithelial cells in a central fiber and fibroblasts in outer fibers (the central and outer fibers being arranged in a sinusoid structure). Alternatively, the outer fibers may be left empty, and the cell-laden constructs can be co-cultured with fibroblasts attached to a suitable support (e.g. the bottom of a culture dish). The fiber-assembled tissue constructs may be used to generate aligned tubules along the longitudinal axis of the fibers (rather than a random network of tubules). In addition, as tubule morphogenesis is promoted in the presence of other cell types such as fibroblasts, the fiber constructs may be used to provide a co-culture environment for the study.

Multi-component fibers of the invention may be utilised in isolation or included in secondary sinusoid units. One non-limiting application of spatially defined multi-component fibers in tissue engineering is the co-culture of cells in their respective niches, within individual multi-component fibers. Such a co-culture configuration may be exploited to achieve optimal cell function. For example, in certain embodiments hepatocytes are cultured to sandwich a middle layer of endothelial cells. The possibility of cell migration and interaction between cell layers within an individual multi-component fiber allows the study of cell to cell interactions in a physiologically relevant three-dimensional environment.

In certain embodiments, multi-component fibers of the invention comprise one or more layers comprising smooth muscle cells and one or more layers comprising endothelial cells.

In other embodiments, multi-component fibers of the invention comprise one or more layers comprising cardiomyocytes and one or more layers comprising endothelial cells.

In other embodiments, multi-component fibers of the invention comprise one or more layers comprising hepatocytes and one or more layers comprising endothelial cells.

In further embodiments, multi-component fibers of the invention comprise one or more layers comprising neurons and one or more layers comprising Schwann cells.

In additional embodiments, multi-component fibers of the invention comprise one or more layers comprising neurons and one or more layers comprising oligodendrocytes.

In other embodiments, multi-component fibers of the invention comprise one or more layers comprising pericytes and one or more layers comprising epithelial cells.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

The invention will now be described with reference to specific examples, which should not be construed as in any way limiting.

### Example 1

### Fiber Production and Assembly into Three-Dimensional Tissue Constructs by Fusion Method

### Materials and Methods

(i) Cell culture

Hepatocellular carcinoma cells (HepG2) were cultured in low glucose DMEM supplemented with 10% fetal bovine serum (FBS) and 1% Penicillin-Streptomycin. HUVECs were cultured in endothelial cell medium (EGM-2; Lonza).

### (ii) Fluorescent Labeling of Cells

HepG2 and HUVEC cells were labeled with Cell Tracker Green (CMFDA; Invitrogen) and Cell Tracker Orange (CMTMR; Invitrogen), respectively, prior to encapsulation by resuspension of the cell pellet in 1 ml of OptiMEM (Invitrogen) with 5 μM of the respective Cell Tracker reagent. The cells were left to incubate at 37° C. for 15 min and centrifuged; the supernatant was removed. The cells were then incubated in 1 ml of OptiMEM for 15 min, followed by phosphate buffered saline (PBS) rinses. Cells used for encapsulation were suspended in their respective culture medium.

(iii) Preparation of Methylated Collagen

Collagen was methylated in this experiment to give it a higher positive charge which allows it to complex with the polyanion solution. Methylated collagen was prepared as follows: 10 ml of 3.75 mg/ml of rat tail collagen (BD Biosciences) was added dropwise to 200 ml of acetone (J. T. Baker) to precipitate collagen. The excess liquid was then decanted, and the collagen was allowed to dry in a dessicator for 3 h. The collagen was then dissolved in 8 ml of 0.1 M HCl, and the resulting solution was added dropwise to a continuously stirred solution of 10 ml of 1 M HCl and 90 ml of methanol (Merck) in order for the methylation to take place. The reaction was allowed to proceed for 24 h, after which the reaction solution was dialyzed against deionized water for 24 h to remove unreacted methanol. The solution was then collected and freeze dried to obtain rat tail methylated collagen (RMC).

### (iv) Preparation of Water-Soluble Chitin

Water-soluble chitin (WSC) was prepared via a modified protocol as described by Sannan et al. (see Sannan et al., (1976), “Studies on chitin 2. Effect of deacetylation on solubility”, Makromolekulare Chemie-Macromolecular Chemistry and Physics 177, 3589-3600).

### (v) Polyelectrolyte Fiber Drawing

To prepare the polycation precursor for HepG2 cells, WSC and RMC were dissolved in PBS at concentrations ranging from 5 mg/ml to 20 mg/ml each. 10 μl of HepG2 cell pellet solution (80-100 million cells/ml) were then added to 90 μl of polycation solution, and mixed thoroughly. A 10 mg/ml solution of sodium alginate in deionized water was used as the polyanion. For the HUVEC fibers, WSC was dissolved in PBS to concentrations ranging from 5 mg/ml to 20 mg/ml. 1 μl of HUVEC cell pellet solution (80-100 million cells/ml) was added to 9 μl of polycation solution. As fibrinogen is negatively charged, it was added to the polyanion solution. Fibrinogen (Sigma) was added to a 10 mg/ml solution of sodium alginate to a final concentration of 37 mg/ml.

5 μl each of polycation and polyanion solutions were dispensed on opposite sides of an adhesive-modified pipette tip, and allowed to come into contact with each other. Fibers were then drawn by an upward motion of the tip by a linear motor at a speed of 0.1 mm/sec.

### (vi) Assembly of Higher Order Constructs

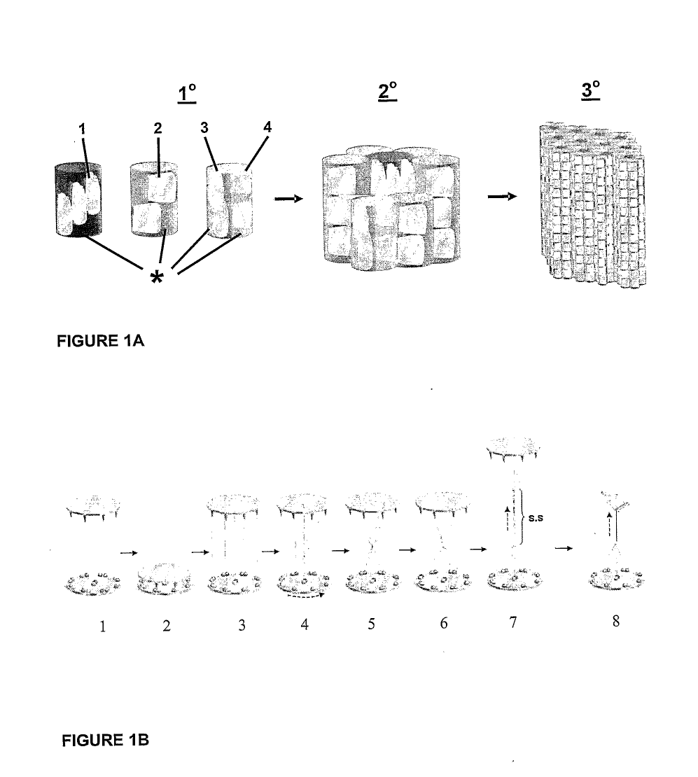

In order to obtain the sinusoid, 9 fibers were drawn in parallel in the configuration (FIG. 1b). The fibers were drawn in a humidified chamber to protect the cells from drying. Once the fibers reached a height of 5 cm, the linear motor was stopped, and the base plate was rotated at 5 rpm for 5 rounds to allow the fibers to meet at a point. A 5-μl droplet of 1 mg/ml sodium alginate was then balanced on the fiber fusion point. Subsequently, the linear motor was reactivated to allow the droplet to travel down the nascent construct and fuse the fibers via secondary complexation. In order to obtain tertiary structures, the sinusoid fiber was spooled on a two-pronged collection rod, and the individual sinusoids were fused by dipping into solutions containing polyelectrolytes (e.g. chitosan, WSC, RMC and alginate) and/or multivalent ions (e.g. Ca²⁺, Fe³⁺). The assembled tertiary structures were then removed from the collecting rod by cutting them with a scalpel at both ends. The constructs were then placed in culture medium.

### Results

The technique was used to assemble fibers containing encapsulated cells as a means to create a micro-patterned 3D environment at high resolution. Each primary biostructural unit (fibers) was composed of a hydrogel matrix that permits the migration and self-assembly of the cells within it. At the same time, the presence of the acellular matrix allows the incorporation of specific cues (e.g. growth factors or extracellular matrix (ECM) components), which are specific for the cells of that particular biostructural unit, to form a niche microenvironment.

The primary biostructural units were subsequently assembled to form a hierarchical construct that offers the potential of providing highly customizable 3D micropatterned environments for growing tissues (FIG. 1a).

The basic biostructural unit produced in the form of a sinusoid is composed of a central endothelial fiber unit wrapped by fibers containing other cell types. To obtain the primary biostructural units, cells were encapsulated into fibers formed by the process of interfacial polyelectrolyte complexation (IPC) between sodium alginate (ALG) and chitin (CHI). These primary units were drawn in parallel from solutions placed on a template, each containing the specific cell type and the niche environment tailored for that particular cell type (FIG. 1b). Rotation of the template brought the fibers to meet at a point, where the fibers were fused by adding a droplet of sodium alginate solution to cause secondary complexation. The upward drawing motion was continued, and subsequent sliding of the alginate droplet down the nascent construct formed the secondary sinusoid structure. This secondary structure was subsequently rolled up to form the tertiary structure.

Each primary biostructural unit is ˜50 μm in diameter. By organizing these primary units into sinusoid structures, every cell in the construct is less than 100 μm away from the central endothelial fiber. The resulting tertiary construct is one that has repeated sinusoid structures with a high density of cells patterned at high resolution (FIG. 1c).

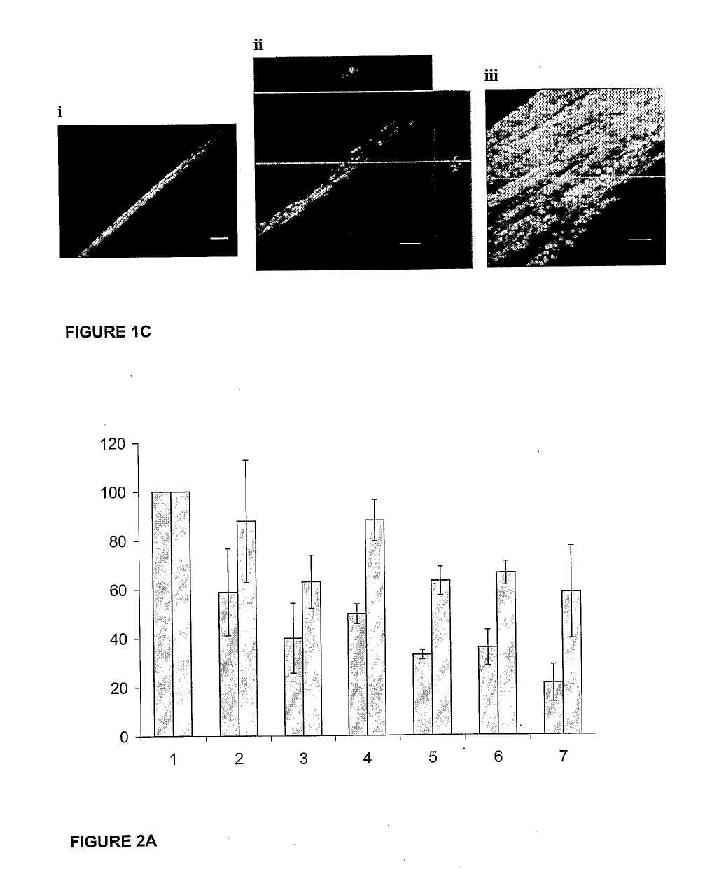

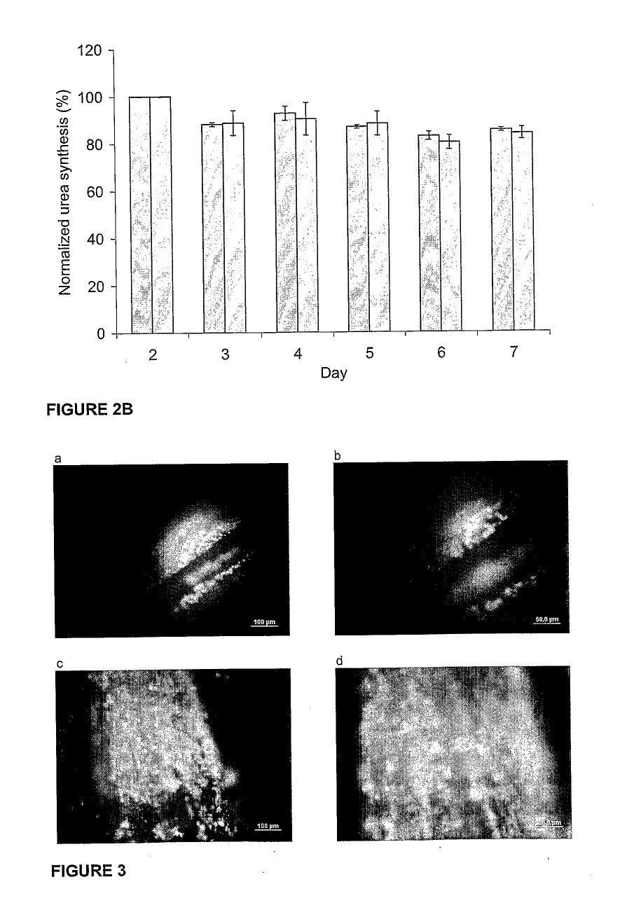

To study the feasibility of using a fiber-assembled tissue construct for hepatic tissue engineering, we fabricated a micropatterned hepatic patch by forming tertiary constructs consisting of human umbilical vein endothelial cells (HUVEC) in the centre fiber and primary rat hepatocytes in the outer fibers on the sinusoid structure. Albumin secretion was measured over a period of 12 days, with sampling conducted every 3 days. The results showed that hepatocytes co-cultured with HUVEC cells exhibited higher protein synthesis function as compared to hepatocytes in single culture (FIG. 2). This indicated that the presence of the HUVEC cells promoted hepatocyte function, providing evidence for cell-cell signaling across fiber barriers within the tertiary construct.

The modular assembly approach allows the individual roles that each fiber-cell unit plays to be assessed separately. To demonstrate the formation of endothelial tubules in the central fiber, HUVEC cells were encapsulated in fibrinogen-based fibers. The construct was fluorescently labeled with Live/Dead cell viability assays at various time points. Good viability of the HUVEC cells was observed for up to 3 days (FIG. 3a-c). At day 3, endothelial tubules could be observed in the fiber (FIG. 3d). This indicated that the microenvironment provided by the fiber allowed for reorganisation of the HUVEC cells, and was suitable for endothelial tubule morphogenesis. Furthermore, as fibrin degradation products are thought to promote angiogenesis in vivo, the fibrinogen localized in the construct is though to promote blood vessel formations. Experimental observations lead to the deduction that the endothelial-fiber unit offered likely anastomosis with the host vasculature when the construct was implanted in vivo.

### Example 2

### Assembly of Fibers into Secondary Structures by Continuous Twisting Method

This method shares similarity with the fusion method of assembling fibers into secondary structures (see Example 1 above). Polyionic solutions were placed on a template. The template was continuously rotated such that the fibers were twisted to form a rope-like structure (see FIG. 14). The fibers were fused together due to secondary complexation resulting from the compression generated by the twisting motion. This method can be used with or without a centre fiber. The minimum number of fibers surrounding the centre fiber is 1.

### Example 3

### Production of Spatially Defined, Multi-Component Fibers by Multi Interfacial Polyelectrolyte Complexation (MIPC)

### Materials and Methods

### (i) Polyelectrolyte Solutions and Chitin

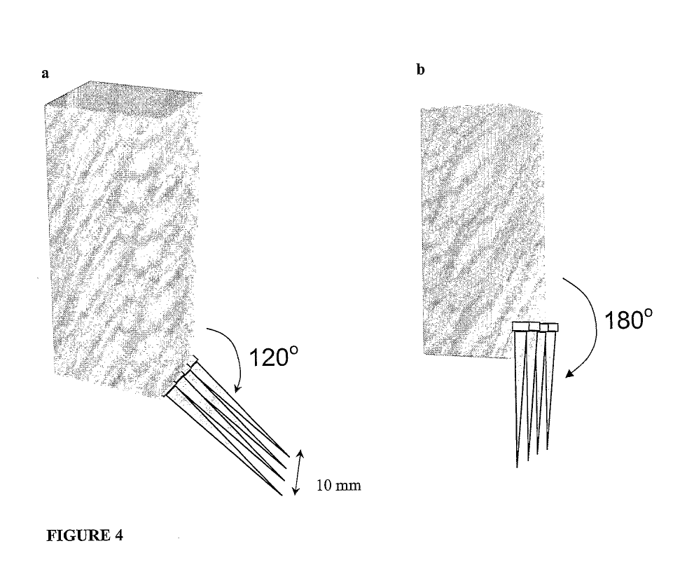

The typical polyelectrolyte solutions used for MIPC fiber drawing were 1% sodium alginate (low viscosity, Sigma) and 0.5 w/v % chitosan (high MW, Aldrich) solution in 2% acetic acid (AR grade, Merck). For a 2-component (binary) fiber, a 2-pipette tip setup (FIG. 2) was employed, whereas a 3-component fiber required a 4-pipette tip setup. (FIG. 3). Water soluble chitin was prepared by a procedure modified from Sannan et al. (see Sannan et al., (1976), “Studies on chitin 2. Effect of deacetylation on solubility”, Makromolekulare Chemie-Macromolecular Chemistry and Physics 177, 3589-3600). Two typical configurations of a linear head-pipette tip apparatus for drawing MIPC fiber is illustrated in FIG. 4.

### (ii) 2-Component Fiber

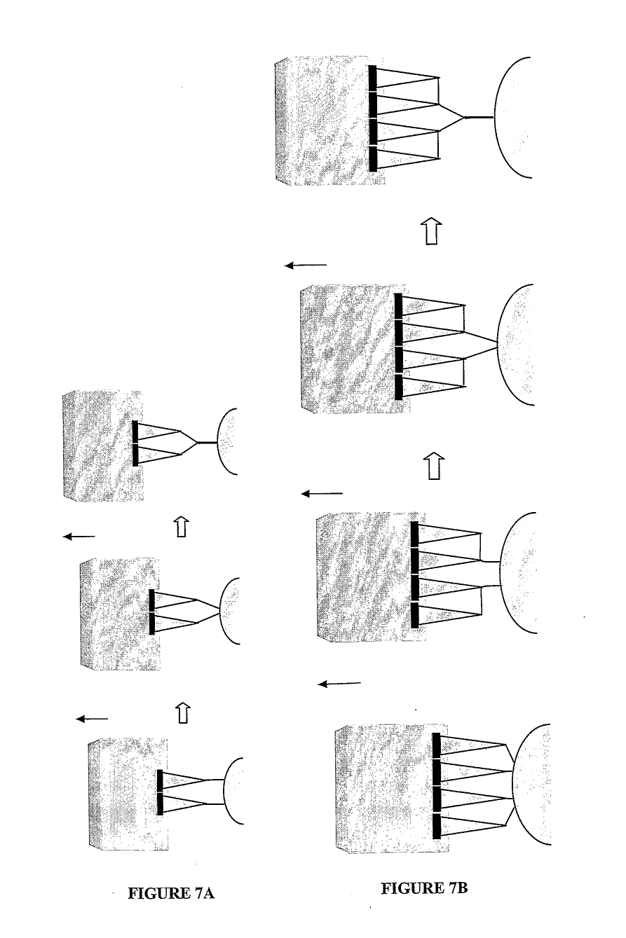

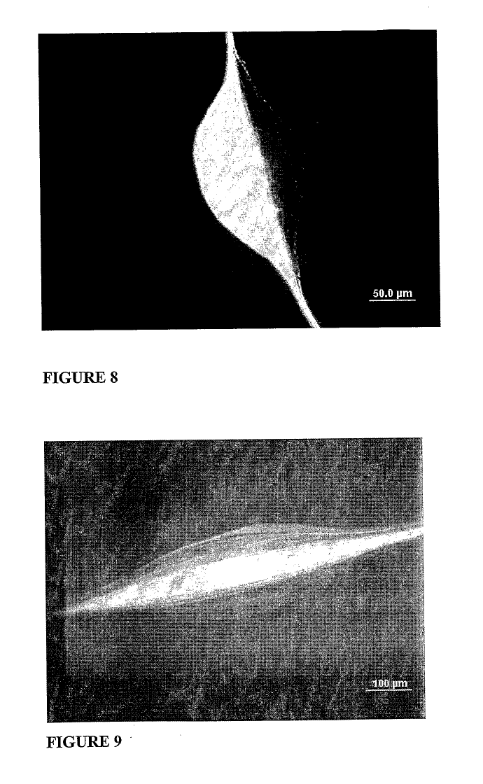

Polyelectrolyte solutions of volume ranging from 5-15 μL were dispensed according to the order shown in FIG. 5a in relation to the pipette tips. The tips were then drawn upwards at a rate of 0.4 mm/s which brought the droplets in contact with one another to form 2 stable interfaces. (FIG. 5b) (Note: if the volume of the solutions were large enough, the solutions would come into contact and form interfaces prior to drawing) Two nascent fibers formed from each interface, which eventually fused to form a single fiber. To obtain the 2-component (binary) MIPC of FIG. 8, the solutions labeled 1 and 3 were 1% alginate solutions containing red and green QDs respectively, and 2 was an unlabelled 0.5% chitosan solution. The typical sequence of fiber drawing and fusion for formation of the 2-component MIPC fiber is illustrated in FIG. 7a.

(iii) 3-Component Fiber

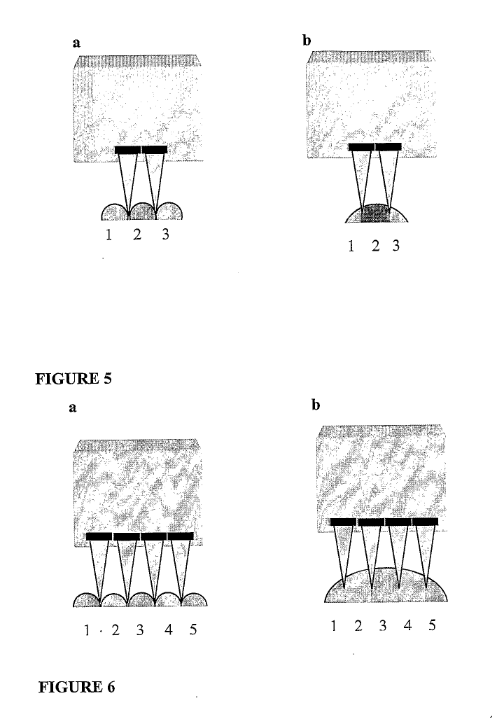

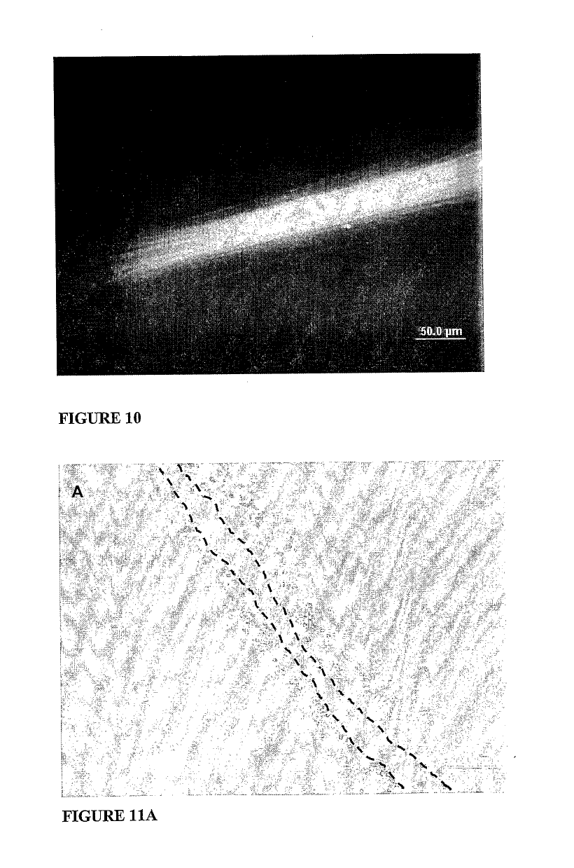

Polyelectrolyte solutions were dispensed in relation to the pipette tips as shown in FIG. 6a. The tips were then drawn upwards at a rate of 0.40 mm/s which brought the droplets in contact with one another to form 4 stable interfaces. (FIG. 6b). To produce the 3-component (ternary) MIPC fibers depicted in FIGS. 9 and 10, the solutions labeled 1, 3 and 5 were 1% alginate solutions containing blue, green and red QDs respectively, while 2 and 4 were unlabelled 0.5% chitosan solution. The typical sequence of fiber drawing and fusion for formation of the 3-component MIPC fiber is illustrated in FIG. 7b. For both 2-component and 3-component MIPC fibers, the sequence and timing of fiber fusion were variable-nevertheless, the overall outcome in terms of fiber structure, composition and spatial definition of components appeared to be similar.

### (iv) MC-3T3 Culture in 3-Component Fiber

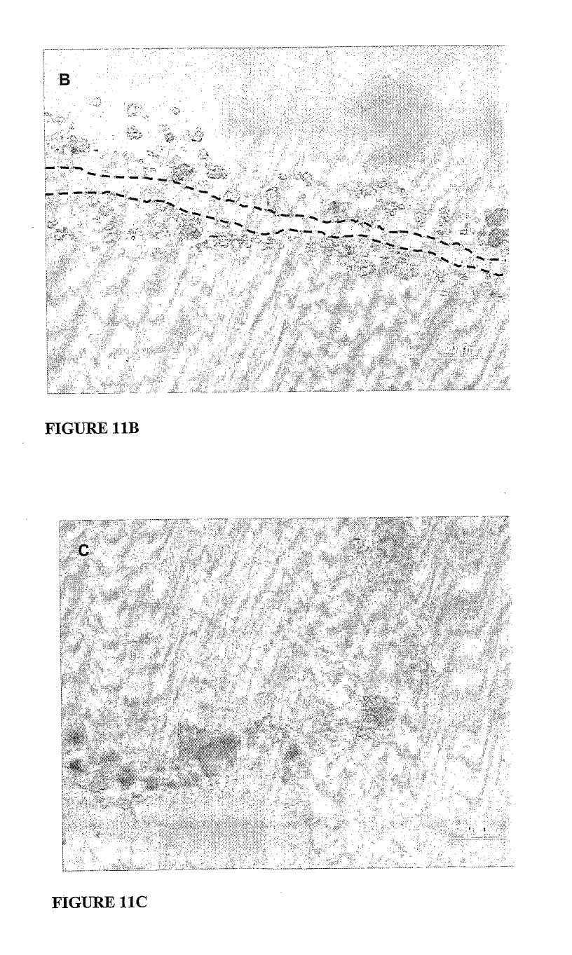

To obtain the 3-component MC-3T3 encapsulated fiber depicted in FIG. 11, three μL of MC3T3 cells from a pellet were added to 30 μL 1% WSC in PBS to constitute solutions 1 and 5 in FIG. 5, while solution 3 was cell-free 1% WSC in PBS. Both solutions 2 and 4 were cell-free 1.0% alginate.

### (v) Primaiy Hepatocytes-Human Umbilical Vascular Endothelial Cell (HUVEC) Culture in 3-Component Fiber

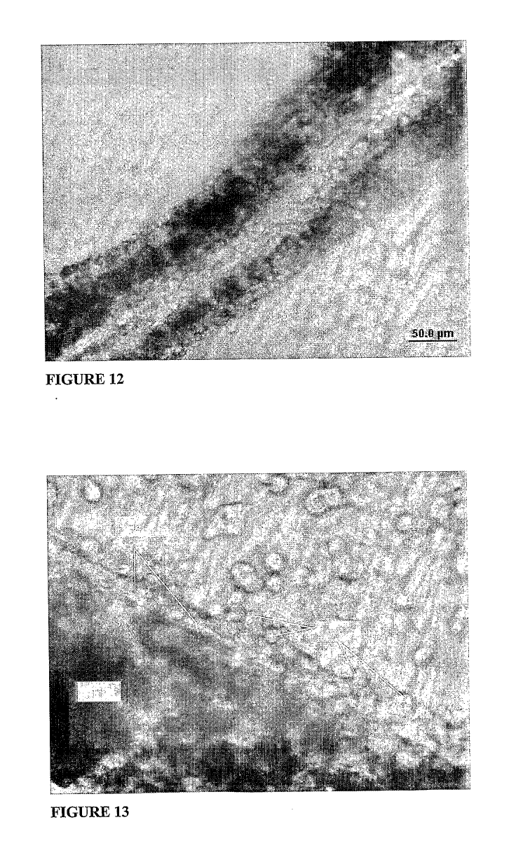

To obtain the 3-component hepatocyte-HLTVEC encapsulated fiber depicted in FIG. 12, 17 μL of primary hepatocytes from a pellet were added to 40 μL 1% WSC, 0.25% rat methylated collagen (RMC) in PBS to constitute solutions 1 and 5, while 4 μL of HUVEC from a pellet was added to 20 μL 1% WSC, 0.25% rat methylated collagen (RMC) in PBS to constitute solution 3. (FIG. 5) Both solutions 2 and 4 were cell-free 1.0% alginate.

The present work provides for an alternative method to pattern cells or other materials within a 3D fiber and construct, with the additional feature of providing for a multi-component fiber whose components are spatially defined within a continuum. These fibers are achieved by simultaneously drawing fiber from multiple interfaces.

### Results and Discussion

The mechanism of IPC fiber formation is thought to involve the process of nuclear fiber formation and coalescence. The method of fiber formation by IPC has been postulated to occur via a multistep mechanism. In the first step a polyelectrolyte complex film is formed at the interface between two oppositely charged PEs, which constitutes a viscous barrier that prevents bulk mixing of the two PEs. When the interface is drawn upwards by a vertical motion, the PE film is broken into separate domains which nucleate further complex formation by consuming PEs from the surrounding solution, forming submicron nuclear fibers. These nuclear fibers are then thought to coalesce to form the primary fiber and beads spaced out at regular intervals along the fiber axis.

The fusion of fibers from two or more interfaces, if it occurred at the point where the nascent fibers leave the solution-air interface, is believed to lead to multiple sets of nuclear fibers, clearly defined in space, within the same primary fiber.

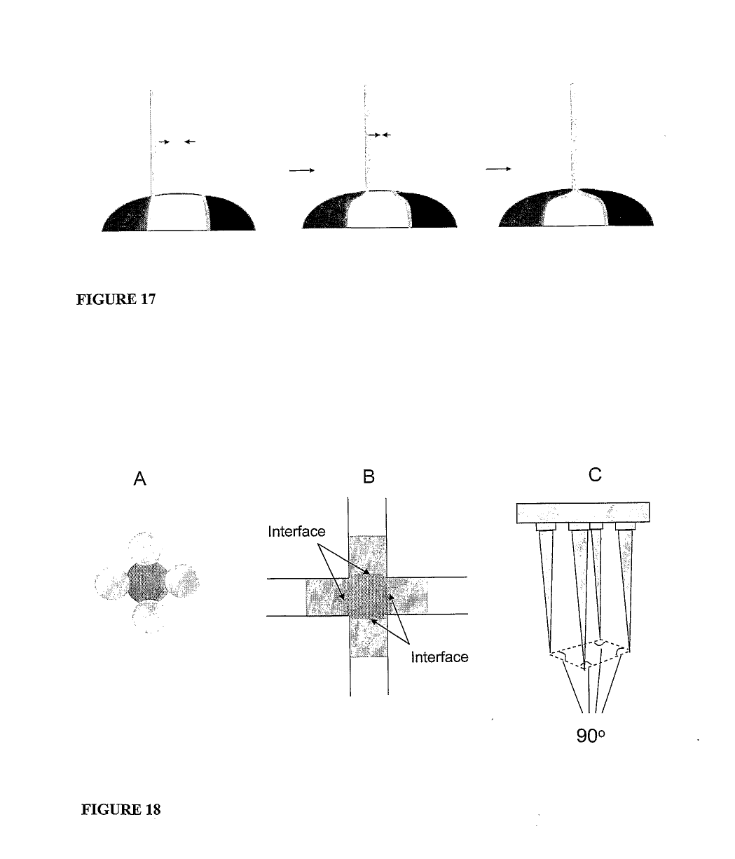

This theoretical insight entailed the practical difficulty of forming two interfaces close enough to allow fusion of the nascent fibers at the solution-air interface within the permissible window of time. To achieve the close proximity of two interfaces, the first experiment employed the configuration depicted in FIG. 5, with a polycation droplet intervening between two polyanion droplets and forming interfacial complexes. As nascent fibers were drawn from the two interfaces, the fibers grew gradually closer to each other due to drifting of the interface or manual action, and eventually fused (FIG. 7a). An interesting observation was the subsequent toggling of the nascent fiber at the solution air interface, something not seen for the case of the typical single interface IPC. The configuration of the fused interface is postulated to assume a configuration very similar to that of a polyelectrolyte multilayer, with alternating polycation and polyanion layers (FIG. 17).

In the same way MIPC fibers can be formed via the fusion of three or more interfaces, simply by adding more PE droplets in series. (FIG. 6) When the number of interfaces exceeds two, an additional consideration is the order in which the nascent fibers fuse. A typical sequence is shown in FIG. 7b.

An alternative configuration for drawing a multi-component fiber is shown in FIG. 18a (plan view). To spatially define the solutions, droplets may be dispensed into channels/grooves as depicted in FIG. 18b. In the example of FIG. 18b, the location of the centre of the interface defines the vertices of a square. The corresponding instrument for drawing the multi-component fiber using the configuration described is shown in FIG. 18c, where the tip ends define the vertices of a corresponding square. Other configurations and corresponding instruments may be possible, for example, where the centre of the interface defines the vertices of a triangle, rectangle, hexagon, octagon or other polygon.

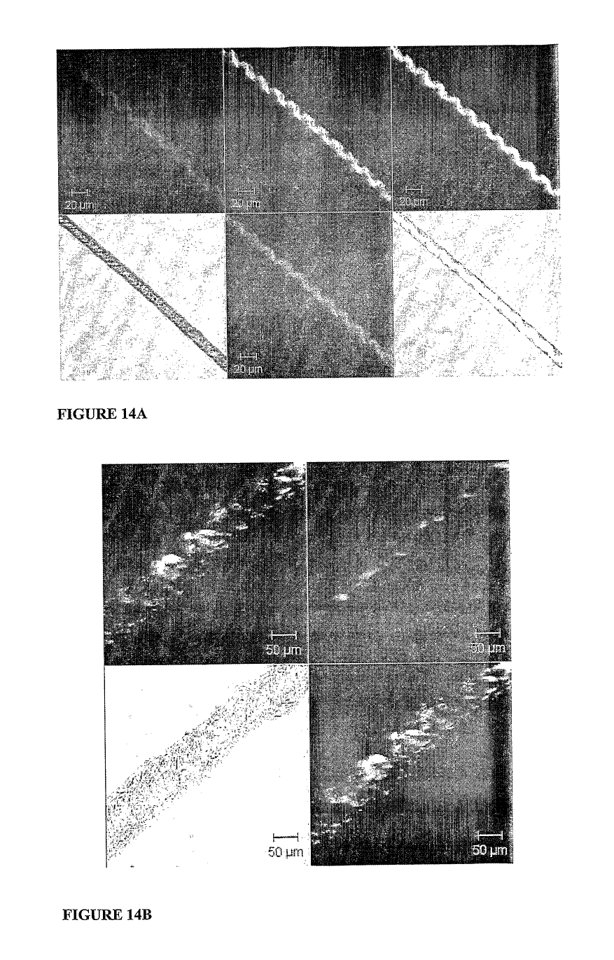

The inunediate application of spatially defined multi component fibers would be the co-culture of cells in their respective niches, within single fibers. Such a co-culture configuration could be exploited to achieve optimal cell function, for example hepatocytes could be cultured to sandwich a middle layer of endothelial cells (FIG. 12). Interaction between the hepatocytes and HUVEC was observed within 24 hours in culture (FIG. 13). The high resolution of 3D cell patterning in such a co-culture would be useful, whether for basic cell biology studies or to fabricate tissue structures for therapeutic purposes.

### Example 4

### Apparatus for Fiber Assembly

An apparatus for the production of fibers in accordance with the invention was manufactured. The machine is shown in FIG. 15, and comprises components including the following:
(a) Fiber drawing template—The template has protruding tips arranged in a pre-determined pattern. The tips are coated with adhesives that allow them to adhere to the fibers drawn from the polyelectrolyte solutions.
(b) Rotating template—The rotating template is where the polyelectrolyte solutions are deposited in a pre-determined pattern. It can be rotated to bring the fibers to meet at a single point. The centre axis of the rotation template is aligned to the centre axis of the fiber drawing template.
(c) Linear motor shaft—Driven by a linear motor, the shaft is attached to the fiber drawing template to draw the fibers upwards continuously.
(d) Humidified box with hinged door—The entire fiber assembly set up is placed inside a humidified box to prevent the fibers from drying up during the process. Humidity inside the box has to be maintained above 60% R.H. for fiber fusion, the fusion droplet to slide down to form the secondary structure and good viability of cells.
(e) Flow inlet for water vapour from humidifier—A constant stream of water vapour is introduced through this inlet to maintain high humidity inside the humidified box. The stream is circulated within the box, but not directed at the fibers as they may cause the fibers to break prematurely.

### Example 5

### Liver Constructs with Aligned Sinusoid Structures

### Materials and Methods

### (i) Reagents

### For Centre Fiber

Polycationic solution—Water soluble chitin (WSC) with methylated rat collagen
Polyanionic solution—Sodium alginate with fibrinogen/vascular endothelial growth factor (VEGF)

### For Outer Fiber

Polycationic solution—Galactosylated water soluble chitin (GWSC) and methylated rat collagen. GWSC is used for niche microenvironment to preserve hepatic function.
Polyanionic solution—Sodium alginate

Endothelial cells were encapsulated in the centre fiber and hepatocytes in the outer fibers to mimic the sinusoid structure. The structure allowed hepatocytes to be co-cultured with the endothelial cells within the same construct in a three-dimensional microenvironment. The microenvironment can also be tailored to maintain the hepatocyte function (e.g. galactose ligands incorporated in the form of galactosylated chitin in the fibers). In addition, the tertiary construct has aligned centre fibers which may contain endothelial cells and angiogenic factors to potentially induce in vivo angiogenesis within the construct.

FIG. 2a indicates that the albumin secretion function of primary rat hepatocytes was better preserved over a period of 7 days when co-cultured with human umbilical vascular endothelial cells (HUVEC) as compared to single culture. The urea synthesis function was also maintained throughout the same period (FIG. 2b).

### Example 6

### Tubule Morphogenesis of Epithelial Cells

Tubule formation is thought to be fundamental to the organization of epithelial cells in organs such as lung, kidney and the reproductive tracts. Further, the formation of tubes is generally more “in vivo-like” in three-dimensional matrices than two-dimensional substrates. However, randomly dispersal of cells are within three-dimensional matrices, has previously resulted in the formation of a random network of tubules. In contrast, the fiber assembly techniques of the present invention can be used to obtain aligned tubules along the longitudinal axis of the fibers. In addition, as tubule morphogenesis is promoted in the presence of other cell types such as fibroblasts, the fiber constructs of the present invention can be used to provide a co-culture environment for the study.

### Materials and Methods

### (i) Reagents

### For Inner Fiber

Polycationic solution—Water soluble chitin and methylated rat collagen
Polyanionic solution—Sodium alginate

### For Outer Fiber

Polycationic solution—Water soluble chitin
Polyanionic solution—Sodium alginate

### Results

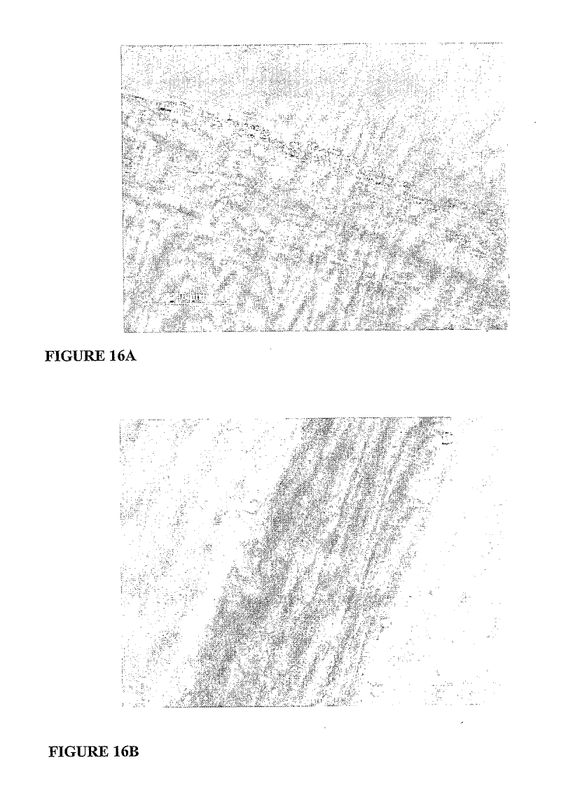

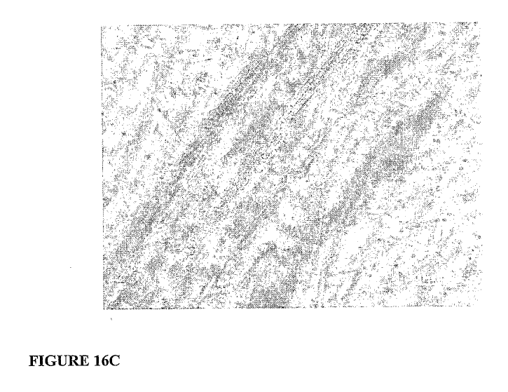

Using these reagents, tubule-forming epithelial cells were encapsulated in the centre fiber and fibroblasts encapsulated in the outer fibers (FIG. 16b). In an alternative experiment the outer fibers were left empty and the cell-laden constructs were co-cultured with fibroblasts attached to the bottom of a culture dish (FIG. 16c). FIG. 16 shows that canine kidney epithelial cells (MDCK) form aligned tubules when co-cultured with NIH/3 T3 fibroblasts.

### Example 7

### Tubule Morphogenesis of Nerve Cells

The fiber assembly technique of the present invention can be used to align neurons in the centre fiber and co-culture neurons with Schwann cells and oligodendrocytes in the outer fibers for regeneration of nerves in the peripheral nervous system and central nervous system, respectively.

1. A fiber-assembled tissue construct comprising at least one sinusoid unit, the unit comprising at least two polymeric fibers arranged in a sinusoid structure by rotating the fibers about a central axis and fusing the fibers together, each of said fibers comprising a porous matrix supporting biological components encapsulated in the fiber, wherein the biological components are patterned in three-dimensions within the construct.

2. The fiber-assembled tissue construct of claim 1, wherein at least one of said biological components is an encapsulated cell.

3. The fiber-assembled tissue construct of claim 1, wherein at least one of said polymeric fibers is a multi-component fiber, said multi-component fiber comprising at least two spatially defined internal domains.

4. The fiber-assembled tissue construct of claim 3, wherein said multi-component fiber comprises a first internal domain and a second internal domain, said first internal domain comprising at least one component that is absent in the second internal domain.

5. The fiber-assembled tissue construct of claim 1, wherein the sinusoid structure comprises a first polymeric fiber and a second polymeric fiber, said first polymeric fiber comprising at least one component that is absent in the second polymeric fiber.

6. The fiber-assembled tissue construct of claim 4, wherein said at least one component is a specific type of cell, biologic or chemical component.

7. The fiber-assembled tissue construct of claim 1, wherein the unit comprises at least one central fiber.

8. The fiber-assembled tissue construct of claim 7, wherein the unit comprises a central fiber wrapped by a plurality of outer fibers.

9. The fiber-assembled tissue construct of claim 1, wherein at least one of said polymeric fibers comprises a biological or chemical component selected from the group consisting of extracellular matrix proteins, cytoskeletal proteins, cell adhesion proteins, hormones, growth factors, angiogenic factors, amino acids, nucleic acids, galactose ligands, drugs, and mixtures thereof.

10. The fiber-assembled tissue construct of claim 1, wherein said sinusoid structure comprises a central fiber, and said central fiber comprises one or more of encapsulated endothelial cells, encapsulated epithelial cells or encapsulated neurons.

11. The fiber-assembled tissue construct of claim 1 wherein the sinusoid structure comprises a central fiber and an outer fiber wrapped around said central fiber, said central fiber comprising encapsulated endothelial cells and said outer fiber comprising encapsulated hepatocytes.

12. The fiber-assembled tissue construct of claim 1, wherein the sinusoid structure comprises a central fiber and an outer fiber wrapped around said central fiber, said central fiber comprising encapsulated epithelial cells and said outer fiber comprising encapsulated fibroblasts.

13. The fiber-assembled tissue construct of claim 1, wherein the sinusoid structure comprises a central fiber and an outer fiber wrapped around said central fiber, said central fiber comprising encapsulated neurons and said outer fiber comprising encapsulated Schwann cells and/or encapsulated oligodendrocytes.

14. A method for producing a three-dimensional fiber-assembled tissue construct comprising at least one sinusoid unit, the method comprising the steps of:
(a) dispensing at least two polyionic solutions in separate locations on a first template;
(b) drawing a separate nascent polymeric fiber from each of said polyionic solutions, wherein a first end of each of said nascent fibers remains attached to the first template and a second end of each of said nascent fibers remains attached to an opposing second template;
(c) rotating either or both templates to contact each of said fibers at a common fusion point; and
(d) fusing contacting fibers together to provide a sinusoid unit, wherein said fusing comprises:
(i) applying a fusing reagent to the fusion point and upwardly drawing each of said fibres such that the reagent travels downwardly along contacting fibers; or
(ii) continuing rotation of either or both templates causing fusion by compressive force.

15. The method of claim 14 comprising the additional step of fusing two or more sinusoid units together.

16. The method of claim 15, wherein said fusing two or more sinusoid units is performed by spooling sinusoid units and fusing them together with a fusing reagent.

17. The method of claim 14, wherein the fusing reagent is selected from the group consisting of polyanionic polymers, polycationic polymers, multivalent cations, multivalent anions, or mixtures thereof.

18. The method of claim 14, wherein at least one of said polymeric fibers is a multi-component fiber, said multi-component fiber comprising at least two spatially defined internal domains.

19. The method of claim 14, wherein the sinusoid unit comprises at least one central fiber and at least one outer fiber wrapped around the central fiber.

20. The method of claim 14, wherein at least one of said polymeric fibers comprises a cell.

21-32. (canceled)
